(19) United States
(12) Patent Application Publication
(10) Pub. No.: US 2013/0006339 A1
(43) Pub. Date: Jan. 3, 2013

(54) IMPLANTABLE BIOMEDICAL DEVICE LEADS COMPRISING LIQUID CONDUCTORS

(76) Inventors: Keith L. March, Carmel, IN (US); William J. Combs, Galena, OH (US); Aaron Matthew Kyle, Teaneck, NJ (US); Nichole Marilyn Leahy-Glass, Indianapolis, IN (US)

(21) Appl. No.: 13/514,926

(22) PCT Filed: Dec. 13, 2010
(86) PCT No.: PCT/US10/60078

§ 371 Date: Sep. 19, 2012

Related U.S. Application Data
(60) Provisional application No. 61/285,706, filed on Dec. 11, 2009.

(51) Int. Cl. A61N 1/05

(52) U.S. Cl. 607/116

(57) ABSTRACT

Implantable biomedical device leads comprising liquid conductors. In an exemplary embodiment of a lead for use in a biomedical application of the present disclosure, the lead comprises a lead body having a distal end and a proximal end, the lead body defining a first interior lumen therethrough, and an electrically conductive composition positioned within the first interior lumen, the electrically conductive composition comprising a metal in a liquid state at or below about 98° F. In another embodiment, the electrically conductive composition is selected from the group consisting of gallium, a gallium-indium alloy, Galinstan, its/their alloys, and combinations thereof. In various embodiments, the metallic electrically conductive composition is used along with a second non-metallic electrically conductive composition such as a conductive polymer, an electrically conductive liquid, an electrically conductive gel, or combinations thereof.

### PRIORITY

The present international patent application is related to, and claims the priority benefit of, U.S. Provisional Patent Application Ser. No. 61/285,706, filed Dec. 11, 2009, the contents of which are hereby incorporated by reference in their entirety into this disclosure.

### BACKGROUND

Heart disease persists as the top cause of death in the United States. There are over 5 million people living with heart disease, with 670,000 new occurrences per year and an annual mortality rate of 266,000. Abnormal rhythm events, or arrhythmias, are often associated with cardiac ischemia or failure. Arrhythmias can by treated by restoring cardiac conduction to a natural rhythm using cardiac rhythm management devices (CRMDs), which administer electrical shocks to the myocardium in response to arrhythmic events.

The most common of CRMDs are cardiac pacemakers and implantable cardioverter defibrillators. Pacemakers are used to restore and maintain synchrony between the sinoatrial node and the ventricles. There are approximately 3 million pacemakers in worldwide use with 600,000 new pacemakers implanted per year. Implantable cardioverter-defibrillators (ICDs) are used to restore normal cardiac electrical function during adverse events such as ventricular tachycardia or fibrillation, and to detect adverse events. ICDs can deliver much higher energy electrical impulses than pacemakers. In response to detected fibrillation, ICDs deliver pulses on the order of joules in order to produce widespread cardiac depolarization and the subsequent restoration of normal rhythm, which is in contrast to the microjoule pulses generated by pacemakers. From 1984 to 2000, the rate of ICD implantation increased from 6/100,000 to 12.7/100,000. Approximately 250,000 ICDs are currently implanted per year.

Conductive leads are used to transmit electrical signals produced by a CRMD to target tissues. Standard lead components are: a connector to interface the lead with the stimulating device; conductor(s) that are typically comprised of some electrically conductive metallic material; insulating materials to shield the conductors from each other and the surrounding environment; and terminating electrode(s) with fixation mechanisms for interface with the tissue. The electrodes and connectors are often made of conducting, inert metals that exhibit minimal polarization effects, e.g., platinum, stainless steel, MP35N alloy, or titanium. Insulators, e.g., silicone rubber, polyurethane, or PTFE, encase the conducting materials, electrically isolating the conductor(s) and protecting the lead's functional components from physiological responses in situ.

Depending upon the nature of the biomedical device, the lead may be required to transmit high power signals, such as the 10-20 A pulse, in the range of 100-1000V typically generated by an implantable cardioverter defibrillator (ICD). The leads may also carry small currents, such as those used in neurological stimulators or cardiac pacemakers, which are on the order of milliamps. Further, implantable biomedical leads may comprise a single, unipolar lead, or they may comprise a bipolar lead, depending upon the therapy to be applied. Regardless of the device's power output, or its unipolar or bipolar configuration, implantable leads are subjected to significant mechanical and chemical stresses in situ. An implantable lead must be able to withstand those harsh circumstances and maintain their ability to conduct the electrical signals necessary to effectively impart treatment to the target tissue.

Cardiac rhythm therapy is ineffective if the signals of interest are not transmitted to and from the device due to lead failure. Leads in situ are subject to a tremendous range of chemical-immunological and mechanical stresses induced by lead deformation that occurs with 37 million heartbeats per year. Modes of lead failure include conductor fracture, insulation fracture, and lead dislodgement at the myocardium. Lead conductors are usually damaged due to excessive bending, torquing, or crushing of the lead at the proximal or distal end. For example, CRMD leads may be implanted between the clavicle and first rib. This position makes the conductors susceptible to compression, creating stress that can eventually lead to breakage. Fracture of conductors presents a serious problem because electrical signals may not be transmitted across the large impedances created by a breakage or the device may sense artifacts created by their mechanical motion or by intermittent contact of the separated but solid conductors. Also, the electrical interference from overlying muscles could be interpreted by ICD sensing circuitry as an arrhythmic event, triggering unnecessary ICD shocks and possibly inducing fibrillation or fatal cardiac arrest.

Several attempts have been made to overcome the fracturing of leads that are subjected to repetitive stress from body motion. A solution proposed in U.S. Pat. No. 3,572,344 to Bolduc for improving mechanical strength of cardiac pacing leads utilizes a twisted platinum strip wire wrapped around a non-conductive core that encapsulates a single wire core. This allows some expansion and contraction of the conductive platinum coils while causing the fiber core to absorb much of the repetitive mechanical shock that to which cardiac leads are subjected. Other attempts to solve the issue of fracturing conductors utilize a central primary conductor cable comprising a central wire or cable helically wrapped by multiple outer conductive wires, such as those described in U.S. Pat. No. 4,640,983 to Comte. It will be appreciated that in many applications, the outer helical wires are comprised of a different conductor composition than that of the central conductor wire or cable. The outer conductor may be a less conductive, but a mechanically stronger material, allowing the many small encapsulating wires to act as an armor plating of sorts that is further able to conduct electrical signals across any small fractures that may develop in the larger, more conductive material below. However, it will be appreciated that such mixing of conductors can result in stresses due to differing physical characteristics as well, and may further add to stresses developed by tightly coiling a harder metal over a softer, weaker, metal or fiber core.

In addition, utilizing cabled or coiled conductors results in significantly longer conductors with significantly smaller conductor cross-sectional areas, thereby increasing the overall resistance of the lead. Electrical resistance of a wire is directly proportional to the wire length and inversely proportional to the square of the wire radius. Thus, as leads become longer or cross-sectional area decreases, the overall lead resistance increases. The increased resistance that occurs with long, small diameter wires may elevate the power requirements or necessitate the use of higher quality, more expensive wire materials to drive the requisite electrical current through these leads. Further, even though the multiple coiled, cabled, or other lead arrangements address the mechanical abuse that implantable biomedical leads must endure, these arrangements do not deal with the corrosive fluids that are ever-present in the tissues and interstitial spaces where the leads must exist, nor do they address the tissue damage that may result from the implantation of biomedical leads.

A biomedical lead that can withstand the various abuses of in vivo utilization would be greatly appreciated.

### BRIEF DESCRIPTION

In an exemplary embodiment of a lead for use in a biomedical application of the present disclosure, the lead comprises a lead body having a distal end and a proximal end, the lead body defining a first interior lumen therethrough, and an electrically conductive composition positioned within the first interior lumen, the electrically conductive composition comprising a metal in a liquid state at or below about 98° F. In another embodiment, the electrically conductive composition is selected from the group consisting of gallium, a gallium-indium alloy, Galinstan, its/their alloys, and combinations thereof. In various embodiments, the metallic electrically conductive composition is used along with a second non-metallic electrically conductive composition such as a conductive polymer, an electrically conductive liquid, an electrically conductive gel, or combinations thereof. In yet another embodiment, the lead body comprises a non-conductive material selected from the group consisting of silicone, polyurethane, polytetrafluoroethylene (PTFE), ethylene tetraflurooethylene (ETFE), polyether ether ketone (PEEK), perfluroralkoxyethylene-tetrafluoroethylene (PFA), fluorinated ethylene propylene (FEP), polyvinylidene fluoride (PVDF), polyamide, parylene, nylon, co-extrusions thereof, and combinations thereof.

In an exemplary embodiment of a lead for use in a biomedical application of the present disclosure, the first interior lumen is filled with the electrically conductive composition under vacuum or under atmospheric pressure. In another embodiment, the electrically conductive composition maintained within the first interior lumen under vacuum or under atmospheric pressure. In an additional embodiment, the electrically conductive composition fills the first interior lumen. In yet an additional embodiment, the lead further comprises a distal component coupled to the lead body at or near the distal end of the lead body, the distal component in conductive communication with the electrically conductive composition. In another embodiment, the distal component is selected from the group consisting of an electrode, a connector, an adapter, a coil, and a closure device. In yet another embodiment, the distal component is capable of sealing the distal end of the lead body to prohibit loss of the electrically conductive composition from the first interior lumen and to mitigate entry of bodily fluids into the first interior lumen.

In an exemplary embodiment of a lead for use in a biomedical application of the present disclosure, the lead further comprises a membrane at least partially surrounding the lead body. In another embodiment, the membrane comprises a biologically-compatible non-conductive material. In yet another embodiment, the lead further comprises a solid conductor positioned within the first interior lumen of the lead body.

In an exemplary embodiment of a lead for use in a biomedical application of the present disclosure, the lead further comprises a solid conductor positioned within the first interior lumen of the lead body. In an additional embodiment, the solid conductor comprises a conductive material selected from the group consisting of stainless steel, platinum, titanium, silver, tantalum, a nickel-cobalt base alloy, its/their alloys, and combinations thereof. In yet an additional embodiment, the electrically conductive composition and the solid conductor are capable of transmitting a first electrical signal therethrough. In another embodiment, the electrically conductive composition and the solid conductor are capable of transmitting the first electrical signal in a first direction. In yet another embodiment, the electrically conductive composition is capable of transmitting a first electrical signal therethrough, and wherein the solid conductor is capable of transmitting a second electrical signal therethrough. In an additional embodiment, the lead further comprises an insulator positioned between the electrically conductive composition and the solid conductor.

In an exemplary embodiment of a lead for use in a biomedical application of the present disclosure, the lead further comprises a second interior lumen defined within the lead body. In an additional embodiment, the lead further comprises a solid conductor positioned within the second interior lumen of the lead body, wherein the electrically conductive composition is capable of transmitting a first electrical signal therethrough, and wherein the solid conductor is capable of transmitting either the first electrical signal or a second electrical signal therethrough. In yet an additional embodiment, the electrically conductive composition is capable of transmitting the first electrical signal in a first direction, and wherein the solid conductor is capable of transmitting the second electrical signal in a second direction. In another embodiment, the solid conductor is selected from the group consisting of an uncoiled wire, a coiled wire, a round wire, a flat wire, a helically turned conductive ribbon, a drawn filled tube wire, a helical hollow strand wire, a drawn brazed strand wire, a cable wire, and a combination thereof.

In an exemplary embodiment of a lead for use in a biomedical application of the present disclosure, the lead further comprises a proximal component coupled to the lead body at or near the proximal end of the lead body, the proximal component in conductive communication with the electrically conductive composition. In another embodiment, the proximal component is selected from the group consisting of an electrode, a connector, and a closure device. In yet another embodiment, the proximal component is capable of sealing the proximal end of the lead body. In an additional embodiment, the lead further comprises a biomedical device coupled to the proximal component, the biomedical device capable of transmitting and/or receiving an electrical signal from the electrically conductive composition. In yet an additional embodiment, the biomedical device is selected from the group consisting of a cardiac pacemaker, an implantable cardioverter defibrillator, an electrocardiogram (EKG or ECG) device, and electroencephalogram (EEG) device, a neurological stimulator, and a non-neurological stimulator.

In an exemplary embodiment of a lead for use in a biomedical application of the present disclosure, the lead further comprises a biomedical device coupled to the lead body at or near the proximal end of the lead body, the biomedical device capable of transmitting and/or receiving an electrical signal from the electrically conductive composition and further capable of sealing the proximal end of the lead body. In an additional embodiment, the lead further comprises a first interior tube positioned within the first interior lumen, the interior tube defining a first interior tube lumen. In yet an additional embodiment, the lead further comprises a solid conductor, wherein the electrically conductive composition is capable of transmitting a first electrical signal therethrough, and wherein the solid conductor is capable of transmitting a second electrical signal therethrough, wherein the electrically conductive composition is positioned within the first interior lumen external to the first interior tube, and wherein the solid conductor is positioned within the first interior tube lumen. In another embodiment, the first interior tube comprises a non-conductive insulator. In yet another embodiment, the electrically conductive composition is positioned within the first interior tube lumen within the first interior lumen.

In an exemplary embodiment of a lead for use in a biomedical application of the present disclosure, the electrically conductive composition is positioned within the first interior lumen external to the first interior tube, and wherein the first interior tube comprises a conductive material. In another embodiment, the first interior tube lumen is configured as a stylet lumen. In yet another embodiment, the lead further comprises a second interior tube positioned within the first interior tube lumen, the second interior tube defining a second interior tube lumen. In an additional embodiment, the electrically conductive composition is positioned within the first interior lumen external to the first interior tube. In yet an additional embodiment, the electrically conductive composition is also positioned within the first interior tube lumen.

In an exemplary embodiment of a lead for use in a biomedical application of the present disclosure, the lead further comprises a solid conductor positioned within the first interior tube lumen, wherein the electrically conductive composition is capable of transmitting a first electrical signal therethrough, and wherein the solid conductor is capable of transmitting a second electrical signal therethrough. In an additional embodiment, the second interior tube lumen is configured as a stylet lumen. In yet an additional embodiment, the first interior tube comprises a non-conductive material, and wherein the electrically conductive composition is positioned within the first interior lumen external to the first interior tube. In another embodiment, the first interior tube lumen is configured as a stylet lumen.

In an exemplary embodiment of a lead for use in a biomedical application of the present disclosure, the lead body further defines a second interior lumen and a third interior lumen. In an additional embodiment, the lead further comprises a first solid conductor positioned within the second interior lumen, wherein the electrically conductive composition is capable of transmitting a first electrical signal therethrough, and wherein the first solid conductor is capable of transmitting a second electrical signal therethrough. In yet an additional embodiment, the third interior lumen is configured as a stylet lumen.

In an exemplary embodiment of a lead for use in a biomedical application of the present disclosure, the lead body further defines a fourth interior lumen. In another embodiment, the electrically conductive composition is also positioned within the second interior lumen and the third interior lumen. In yet another embodiment, the electrically conductive composition is also positioned and the fourth interior lumen. In an additional embodiment, the lead further comprises a second solid conductor positioned within the third interior lumen, the second solid conductor capable of transmitting a third electrical signal therethrough. In yet an additional embodiment, one or more of the first electrical signal, the second electrical signal, and the third electrical signal are equivalent.

In an exemplary embodiment of a lead for use in a biomedical application of the present disclosure, the lead body further defines a second interior lumen. In an additional embodiment, the electrically conductive composition is also positioned within the second interior lumen. In yet an additional embodiment, the lead further comprises a first solid conductor positioned within the first interior lumen, wherein the electrically conductive composition is capable of transmitting a first electrical signal therethrough, and wherein the first solid conductor is capable of transmitting a second electrical signal therethrough. In another embodiment, the lead further comprises a second solid conductor positioned within the second interior lumen, the second solid conductor capable of transmitting a third electrical signal therethrough. In yet another embodiment, the lead further comprises a first solid conductor capable of transmitting a second electrical signal therethrough, the first solid conductor positioned within the second interior lumen.

In an exemplary embodiment of a lead for use in a biomedical application of the present disclosure, the lead further comprises a doped portion at or near the distal end of the lead body, the doped portion comprising a non-conductive material and a conductive material. In another embodiment, the conductive material is selected from the group consisting of stainless steel, platinum, titanium, silver, tantalum, a nickel-cobalt base alloy, carbon black, carbon fiber, nanofiber tubes, polyaniline, polypyrrole, polythiophene, polyethylenedioxythiophene, and poly(p-phenylene vinylene). In an additional embodiment, the lead further comprises a cuff positioned at least circumferentially around the lead body, the cuff configured to compress at least a portion of the lead body to facilitate coupling of the distal component to the lead body. In yet an additional embodiment, the solid conductor is coated with an insulated coating.

In an exemplary embodiment of a lead for use in a biomedical application of the present disclosure, the distal component defines a circumferential groove separating an outer portion from an inner portion, and wherein the circumferential groove is configured so that the distal end of the lead body fits within the circumferential groove so that the outer portion is on the relative outside of the lead body and so that the inner portion is within the first interior lumen. In an additional embodiment, the distal component is sized and shaped as a pin, the pin having an outer perimeter sized and shaped to fit within the first interior lumen. In yet an additional embodiment, the distal component is configured to engage an external electrode.

In an exemplary embodiment of a lead for use in a biomedical application of the present disclosure, the lead body has a cross-sectional area configured so that one or more conductance pockets are defined within the first interior lumen upon compression of the lead body. In another embodiment, the lead body comprises one or more relatively thinner portions and one or more relatively thicker portions, wherein said one or more relatively thinner portions and the one or more relatively thicker portions define an irregularly-shaped first interior lumen. In yet another embodiment, when the lead body is compressed, the one or more relatively thicker portions define one or more conductance pockets therein.

In an exemplary embodiment of a lead for use in a biomedical application of the present disclosure, the lead further comprises at least two solid conductors positioned within the first interior lumen. In an additional embodiment, the electrically conductive composition and the at least two solid conductors are capable of transmitting a first electrical signal. In yet an additional embodiment, each of the at least two solid conductors are surrounded by an insulator. In another embodiment, the electrically conductive composition is capable of transmitting a first electrical signal, and wherein the at least two solid conductors are capable of transmitting a second electrical signal.

In an exemplary embodiment of a lead for use in a biomedical application of the present disclosure, the first interior lumen is configured as a strain relief lumen. In an additional embodiment, the lead further comprises one or more strain relief lumens defined within the lead body, the one or more strain relief lumens configured so that upon compression of the lead body, the one or more strain relief lumens compresses before the first interior lumen. In yet an additional embodiment, the lead is capable of transmitting an electrical signal through the electrically conductive composition at a lower resistance than a traditional lead having equivalent dimensions but using only a solid conductor. In another embodiment, the lead is capable of transmitting an electrical signal through the electrically conductive composition resulting in less signal energy loss in lead impedance through the electrically conductive composition as compared to signal energy losses in lead impedance through a solid conductor of a traditional lead. In yet another embodiment, the signal energy losses are due to conduction (i2R) losses through the various leads. In an additional embodiment, the first interior lumen comprises a configuration selected from the group consisting of a straight configuration, a twisted configuration, and a coiled configuration.

In an exemplary embodiment of a lead for use in a biomedical application of the present disclosure, the lead is operable as a unipolar lead. In another embodiment, the lead is operable as a bipolar lead. In yet another embodiment, the lead further comprises a second interior lumen and a third interior lumen defined by the lead body, the second interior lumen and the third interior lumen each having the electrically conductive composition and/or a solid conductor positioned therethrough. In an additional embodiment, the lead is operable as a multipolar lead. In yet an additional embodiment, the electrically conductive composition is biologically-compatible. In another embodiment, the electrically conductive composition is non-toxic.

In an exemplary embodiment of a lead for use in a biomedical application of the present disclosure, the first interior lumen has a diameter of between about 0.005″ and about 0.108″. In another embodiment, the lead body has a wall thickness of between about 0.005″ and about 0.054″. In yet another embodiment, the lead body has an outer diameter of between about 1 French to about 12 French. In an additional embodiment, the lead body has a length between about 10 cm and about 200 cm.

In an exemplary embodiment of a lead for use in a biomedical application of the present disclosure, the distal component is at least partially coated with a fibrosis-enhancing substance, the fibrosis-enhancing substance capable of facilitating localized fibrosis at or near the distal component upon connection of the lead to a tissue within a patient's body to reduce the likelihood of unintended disconnection of the lead from the tissue. In an additional embodiment, the fibrosis-enhancing substance is selected from the group consisting of an irritant, polylysine, high-concentration lysine peptides, growth factors, inflammatory cytokines, activin, and angiopoietin. In yet an additional embodiment, the distal component is at least partially coated with a fibrosis-reducing substance, the fibrosis-reducing substance capable of inhibiting localized fibrosis at or near the distal component upon implantation of the lead within a patient's body to reduce a likelihood of a loss or reduction of an electrical signal through the lead. In another embodiment, the fibrosis-reducing substance comprises dexamethasone.

In an exemplary embodiment of a lead for use in a biomedical application of the present disclosure, the lead body comprises a material capable of flexion so that repeated bending of the lead body does not result in loss of conductance through the electrically conductive material. In another embodiment, the electrically conductive composition is sufficiently pliable so to reduce a likelihood of fracture of the lead body at a location where at least a portion of the electrically conductive composition contacts at least a portion of the lead body. In yet another embodiment, the lead body is sufficiently flexible and the electrically conductive composition is sufficiently pliable so to reduce a likelihood of tissue perforation within a patient's body when the lead is positioned within the patient's body and coupled to the tissue as compared to a traditional lead having a solid conductor.

In an exemplary embodiment of a lead for use in a biomedical application of the present disclosure, the lead further comprises a second conductor composition positioned within the first interior lumen of the lead body. In another embodiment, the first electrically conductive composition comprises a metal in a liquid state selected from the group consisting of gallium, a gallium-indium alloy, Galinstan, its/their alloys, and combinations thereof, and wherein the second conductor composition comprises a non-metallic composition selected from the group consisting of a conductive polymer, an electrically conductive liquid, an electrically conductive gel, and combinations thereof.

In an exemplary embodiment of a system of the present disclosure, the system comprises a lead for use in a biomedical application of the present disclosure, comprising a lead body having a distal end and a proximal end, the lead body defining a first interior lumen therethrough, and an electrically conductive composition positioned within the first interior lumen, the electrically conductive composition comprising a metal in a liquid state at or below about 98° F., and a biomedical device coupled to the lead, the biomedical device capable of transmitting and/or receiving an electrical signal from the electrically conductive composition within the lead.

In an exemplary embodiment of a lead for use in a biomedical application of the present disclosure, the lead comprises a lead body having a distal end and a proximal end, the lead body defining a first interior lumen therethrough; and an electrically conductive composition positioned within the first interior lumen, the electrically conductive composition in a liquid or gel state at or below about 98° F. In another embodiment, the electrically conductive composition comprises a metal in a liquid state selected from the group consisting of gallium, a gallium-indium alloy, Galinstan, its/their alloys, and combinations thereof. In yet another embodiment, the electrically conductive composition is selected from the group consisting of gallium, a gallium-indium alloy, Galinstan, its/their alloys, a conductive polymer, an electrically conductive liquid, an electrically conductive gel, and combinations thereof. In an additional embodiment, wherein the electrically conductive composition comprises a first electrically conductive composition and a second electrically conductive composition. In yet an additional embodiment, the first electrically conductive composition comprises a metal in a liquid state selected from the group consisting of gallium, a gallium-indium alloy, Galinstan, its/their alloys, and combinations thereof, and wherein the second electrically conductive composition comprises a non-metallic composition selected from the group consisting of a conductive polymer, an electrically conductive liquid, an electrically conductive gel, and combinations thereof.

In an exemplary embodiment of a system of the present disclosure, the system comprises a lead for use in a biomedical application of the present disclosure, comprising a lead body having a distal end and a proximal end, the lead body defining a first interior lumen therethrough, and an electrically conductive composition positioned within the first interior lumen, the electrically conductive composition in a liquid or gel state at or below about 98° F., and a biomedical device coupled to the lead, the biomedical device capable of transmitting and/or receiving an electrical signal from the electrically conductive composition within the lead.

In an exemplary embodiment of a lead for use in a biomedical application of the present disclosure, the lead comprises a lead body having a distal end and a proximal end, the lead body defining a first interior lumen therethrough, an electrically conductive composition positioned within the first interior lumen, the electrically conductive composition comprising a metal in a liquid state at or below about 98° F., the electrically conductive composition selected from the group consisting of gallium, a gallium-indium alloy, Galinstan, its/their alloys, and combinations thereof, a distal component coupled to the lead body at or near the distal end of the lead body, the distal component in conductive communication with the electrically conductive composition, the distal component capable of sealing the distal end of the lead body, and a proximal component coupled to the lead body at or near the proximal end of the lead body, the proximal component in conductive communication with the electrically conductive composition, the proximal component capable of sealing the proximal end of the lead body, the lead capable of transmitting a first electrical signal from the proximal component, through the electrically conductive composition, and to the distal component.

In an exemplary embodiment of a system of the present disclosure, the system comprises a lead for use in a biomedical application of the present disclosure, comprising a lead body having a distal end and a proximal end, the lead body defining a first interior lumen therethrough, and an electrically conductive composition positioned within the first interior lumen, the electrically conductive composition comprising a metal in a liquid state at or below about 98° F., the electrically conductive composition selected from the group consisting of gallium, a gallium-indium alloy, Galinstan, its/their alloys, and combinations thereof, a distal component coupled to the lead body at or near the distal end of the lead body, the distal component in conductive communication with the electrically conductive composition, the distal component capable of sealing the distal end of the lead body, and a proximal component coupled to the lead body at or near the proximal end of the lead body, the proximal component in conductive communication with the electrically conductive composition, the proximal component capable of sealing the proximal end of the lead body, the lead capable of transmitting a first electrical signal from the proximal component, through the electrically conductive composition, and to the distal component, and a biomedical device coupled to the lead, the biomedical device capable of transmitting and/or receiving an electrical signal from the electrically conductive composition within the lead.

In an exemplary embodiment of a lead for use in a biomedical application of the present disclosure, the lead body having a distal end and a proximal end, the lead body defining a first interior lumen therethrough, and the lead body configured to contain an electrically conductive composition within the first interior lumen, the electrically conductive composition in a liquid or gel state at or below about 98° F., the lead capable of transmitting a first electrical signal through the electrically conductive composition.

In an exemplary embodiment of a system of the present disclosure, the system comprises an exemplary lead of the present disclosure and a biomedical device coupled to the lead, the biomedical device capable of transmitting and/or receiving an electrical signal from the electrically conductive composition within the lead.

### BRIEF DESCRIPTION OF THE DRAWINGS

FIG. 1A shows a perspective side view of an exemplary lead according to at least one embodiment of the present disclosure;

FIG. 1B shows a side view of an exemplary lead according to at least one embodiment of the present disclosure;

FIGS. 2A-2C show cross-sectional views of exemplary leads according to various embodiments of the present disclosure;

FIGS. 3A and 3B show side views of exemplary leads according to various embodiments of the present disclosure;

FIG. 4A shows a perspective side view of an exemplary lead according to at least one embodiment of the present disclosure;

FIG. 4B shows a side view of an exemplary lead according to at least one embodiment of the present disclosure;

FIGS. 5A, 5B, and 6A show cross-sectional views of exemplary leads according to various embodiments of the present disclosure;

FIG. 6B shows a side view of an exemplary lead according to at least one embodiment of the present disclosure;

FIGS. 6C shows a cross-sectional view of an exemplary lead according to at least one embodiment of the present disclosure;

FIGS. 6D, 7A, and 7B show side views of exemplary leads according to various embodiments of the present disclosure;

FIGS. 8A-10J show cross-sectional views of exemplary leads according to various embodiments of the present disclosure;

FIGS. 11A-11C show perspective side views of exemplary leads having a distal component coupled thereto according to various embodiments of the present disclosure;

FIG. 12 shows a graph showing the voltage measure across an exemplary lead according to at least one embodiment of the present disclosure;

FIG. 13 shows a graph showing the measured step response of an exemplary lead according to at least one embodiment of the present disclosure;

FIG. 14A shows an exemplary set of schematics of circuits used to measure the effects of an exemplary lead according to at least one embodiment of the present disclosure;

FIG. 14B shows pulse measurement with a conventional lead and with an exemplary lead according to at least one embodiment of the present disclosure;

FIG. 15A shows a graph showing pulse measurements with a conventional lead cross-correlated with pulse measurements of a conventional lead in series with an exemplary lead according to at least one embodiment of the present disclosure;

FIG. 15B shows a graph showing power spectral density estimates from a pacemaker with a conventional lead and with a conventional lead in series with an exemplary lead according to at least one embodiment of the present disclosure;

FIGS. 16A and 16B show microscopic images of cells incubated with an electrically conductive composition used in connection with exemplary leads according to various embodiments of the present disclosure;

FIG. 17A shows a chart displaying mean cell counts from cells exposed to an electrically conductive composition used in connection with exemplary leads of the present disclosure as compared to controls;

FIG. 17B shows a chart displaying hemocompatibility assay results obtained from testing an exemplary electrically conductive composition of the present disclosure,

FIG. 18 shows a cardiac electrogram obtained using a exemplary lead of the present disclosure.

### DETAILED DESCRIPTION

The present application discloses various implantable leads and methods for using and constructing the same. For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

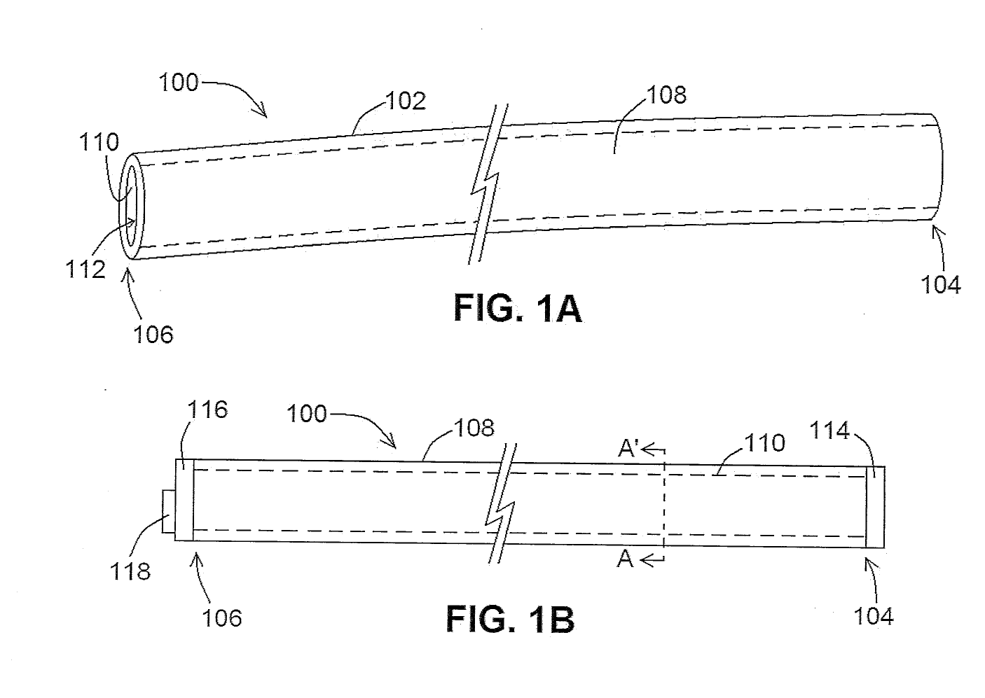

According to at least one embodiment of an implantable biomedical device lead of the present disclosure, and as shown in FIG. 1A, lead 100 comprises a lead body 102 having a proximal end 104 and a distal end 106. Lead body 102, in various embodiments, defines a first outer surface 108 and a first inner surface 110, wherein the first outer surface 108 is on the relative outside of lead body 102, and wherein the first inner surface 110 is the opposing inner surface of first outer surface 108. In such an embodiment, lead body 102 has a tubular shape, defining a first interior lumen 112 therein. As referenced herein, the term “lumen” is defined as a portion within a lead 100 configured to optionally receive and retain a conductor, such as an electrically conductive composition 200 referenced in further detail herein, and is not limited to traditional tubular lumens.

Leads 100 of the present disclosure comprise various components at or near the proximal ends 104 and the distal ends 106 of said leads. In at least one embodiment, and as shown in FIG. 1B, lead 100 comprises a proximal component 114 at or near the proximal end 104 of lead body 102, and a distal component 116 at or near a distal end 106 of lead body 102. Components 114, 116, in various embodiments, may comprise electrodes, connectors, coils, closure devices, etc., useful not only to seal a/the respective end(s) 104, 106 of said lead body 102, but also, in various embodiments, to prevent bodily fluids from entering lumen 112, to perform a function (such as an electrode), to engage a tissue (such as a coil), or to facilitate connection of lead 100 to another component, for example. The jagged lines present in the relative centers of leads 100 shown in FIGS. 1A and 1B are intended to indicate that leads 100 of the present disclosure can have various lengths.

Lead body 102 may comprise any number of biologically-compatible non-conductive or conductive components useful for implantable leads. In various embodiments, lead bodies 102 comprise silicone and/or polyurethane. In other embodiments, for example, lead bodies 102 (as well as insulators 902, referenced in further detail herein), may comprise, but are not limited to, polytetrafluoroethylene (PTFE), ethylene tetraflurooethylene (ETFE), polyether ether ketone (PEEK), perfluroralkoxyethylene-tetrafluoroethylene (PFA), fluorinated ethylene propylene (FEP), polyvinylidene fluoride (PVDF), polyamide, parylene, nylon, or any number of co-extrusions of said polymers or combinations of said polymers.

In addition, portions of various leads 100 of the present disclosure may be coated with a substance 118 to facilitate localized fibrosis. For example, and as shown in FIG. 1B, distal component 116 could optionally have a fibrosis-enhancing substance 118, such as a steroid, positioned thereon so that local fibrosis at the location of adherence of distal component 116 to a heart would be expedited, as such localized fibrosis can help maintain a secure connection of lead 100 to the heart. Various embodiments of such a fibrosis-enhancing substance 118 is selected from the group consisting of an irritant, polylysine, high-concentration lysine peptides, growth factors, inflammatory cytokines, activin, and angiopoietin. In other embodiments, distal component 116 is at least partially coated with a fibrosis-reducing substance 118, such as dexamethasone, for example, which is capable of inhibiting localized fibrosis at or near distal component 116 upon implantation of lead 100 within a patient's body to reduce a likelihood of a loss or reduction of an electrical signal through lead 100.

Leads 100 of the present disclosure may be relatively long and narrow. For example, various leads 100 of the present disclosure may have a lead body 102 thickness of about 0.005″ (or even less) to about 0.108″, and lead 100 outer diameters as large as about 12 French (about 0.118″). Various leads may have lumen diameters as small as about 0.0165″, or within a range of about 0.005″ to about 0.108″. For example, various leads 100 of the present disclosure may have outer diameters between about 1 French to about 12 French. Furthermore, various leads 100 of the present disclosure may have various lead body 102 lengths. In at least one embodiment, lead body 102 has a length of approximately 50 cm. In other embodiments, lead bodies 102 may have lengths between 10 cm and 200 cm. Various other lumen 112 diameters, lead body 102 thicknesses, lead 100 outer diameters, and lead body 102 lengths may be present in any number of leads 100 of the present disclosure, as the exemplary sizes and ranges provided herein are merely exemplary in nature, noting that various lead 100 embodiments may have dimensions outside of said ranges.

In various embodiments, and as shown in FIG. 1B, an exemplary embodiment of a lead 100 of the present disclosure may comprise a first electrode (an exemplary proximal component 114) in sealable contact with the proximal end 104 of lead body 102, and may further comprise a second electrode (an exemplary distal component 116) in sealable contact with the distal end 106 of lead body 102.

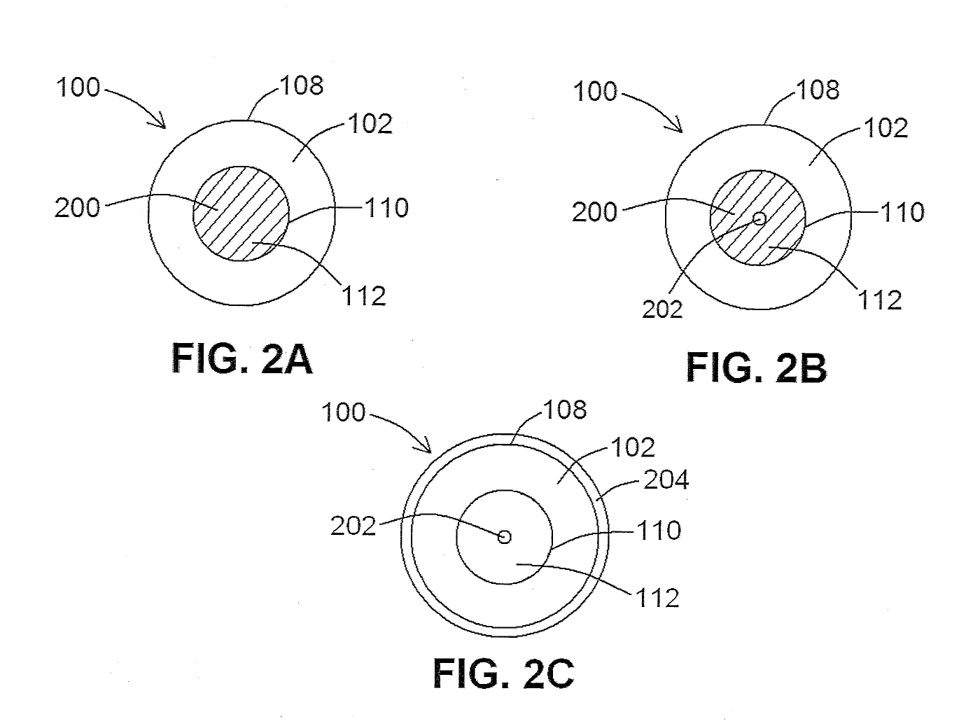

FIG. 2A shows a cross-sectional view of an exemplary lead 100 of the present disclosure along section A-A′ as shown in FIG. 1B. As shown in FIG. 2A, operable leads 100 of the present disclosure comprise an electrically conductive composition 200 present within first interior lumen 112 and/or another lumen as may be the case with one or more embodiments of leads 100 of the present disclosure. Electrically conductive composition 200, as shown in FIG. 2A, may be depicted as a series of diagonal lines. Various exemplary electrically conductive compositions 200 of the present disclosure may comprise liquid metal, such as, for example, gallium, a gallium-indium alloy, Galinstan, its/their alloys, and combinations thereof, having a melting point at or below 98° F. (approximate mammalian body temperature) or otherwise being a liquid or a gel at or below 98° F. Various other electrically conductive compositions 200 of the present disclosure may comprise non-metallic electrically conductive polymers, liquids, and/or gels, having a melting point at or below 98° F. or otherwise being a liquid or a gel at or below 98° F. Various embodiments of leads 100, using electrically conductive compositions 200 of the present disclosure, are operable for use in various sensing and pacing applications as referenced herein.

Furthermore, and depending on the material used to produce tube body 102 (or other non-conductive and/or insulative bodies described herein that come in direct contact with electrically conductive composition 200), electrically conductive composition 200 may readily disengage itself from tube body 102, or may tend to remain in contact with tube body 102, the latter generally referred to as a wetting property of electrically conductive composition 200. Wetting may be desired or undesired depending upon application, and as such, suitable materials may be selected to produce tube body 102, for example, to correspond with said desired application.

Electrically conductive compositions 200 provide a means of conducting a signal/electricity within lead 100. For example, a lead 100 of the present disclosure as shown in FIG. 1B may have an electrically conductive composition 200 positioned within lumen 112 so that electrically conductive composition 200 is capable of conducting a signal/electricity between proximal component 114 and distal component 116. As shown in FIG. 1B, the dotted lines represent the first inner surface 110 of lead 100.

Various other embodiments of leads 100 of the present disclosure, and as shown in FIG. 2B, may comprise a conductor wire 202 positioned within the first interior lumen 112 of lead 100 or within another portion/component of lead 100. Conductor wire 202, in embodiments of leads having at least one conductor wire 202, may provide another means of conducting a signal/electricity within lead 100 along with an electrically conductive composition 200 positioned within said lead 100, thereby creating a redundancy of the conductive properties of lead 100 and providing additional rigidity to lead 100. This exemplary redundancy and rigidity help to prevent lead body 102 from kinking and causing a separation between any of the electrically conductive composition 200 that would preclude conduction of any therapeutic electrical impulses. In the event of such a kink which could result in the collapse of interior lumen 112, conductor wire 202 would maintain conduction between the separated portions of electrically conductive composition 200. Conductor wire 202, in various embodiments, may be any solid conductor currently used in connection with various biomedical leads, and may comprise uncoiled wire, a coiled wire, a round wire, a flat wire, a helically turned conductive ribbon, a drawn filled tube wire, a helical hollow strand wire, a drawn brazed strand wire, a cable wire, a combination thereof, or another suitable solid conductor. In addition, conductor wire 202 (also referred to as a solid metal component/layer 904 referenced in further detail herein) may have a coating thereon, such as an insulator 902 described in further detail herein, so that electrically conductive composition 200 and conductor wire 202/solid metal component/layer 904 can carry separate signals.

FIG. 2C shows another cross-sectional view of an exemplary lead 100 of the present disclosure. Lead 100, as shown in FIG. 2C, shows the same components as the cross-sectional view of lead 100 shown in FIG. 2B, but without the electrically conductive composition 200 included therein. Such an embodiment, for example, could be filled with an electrically conductive composition 200 to produce a lead 100 as shown in FIG. 2B. In addition, the exemplary embodiment of lead 100 shown in FIG. 2C comprises a membrane 204 surrounding at least part of lead body 102. In such an embodiment, membrane 204 may be used to insulate a conductive portion of lead 100 from a bodily tissue, for example. In various embodiments of leads 100 comprising membrane 204, membrane 204 may comprise any number of biologically-compatible non-conductive materials.

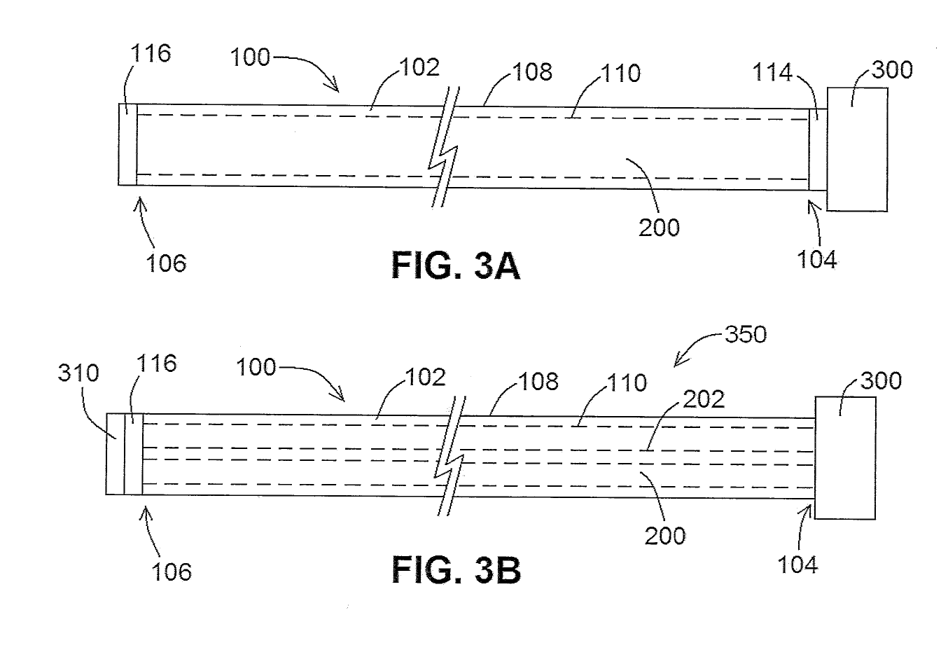

FIGS. 3A and 3B show exemplary embodiments of a lead 100 and a system of the present disclosure comprising an exemplary lead. As shown in FIG. 3A, lead 100 comprises a proximal component 114 coupled to lead 100 at or near the proximal end 104 of lead 100, and comprises a distal component 116 coupled to lead 100 at or near the distal end 106 of lead 100. In addition, and as shown in FIG. 3A, lead 100 is coupled to a biomedical device 300, with said coupling occurring between biomedical device 300 and proximal component 114. FIG. 3A may be viewed as a lead 100 coupled to a biomedical device 300, or may be considered as a system 350 of the present disclosure, wherein system 300 comprises a lead 100 of the present disclosure and some other component, such as, for example, biomedical device 300.

In such an embodiment, and as shown in FIG. 3A, a signal and/or electricity may be transmitted to or from biomedical device 300 to a proximal component 114, through an electrically conductive composition 200 present within lead body 102, and to a distal component 116. In such an embodiment, for example, proximal component 114 may be a connector or an adapter (or component of a connector or adapter) to connect lead 100 to biomedical device 300, and distal component 116 may be an electrode, a connector, a coil, and/or a closure device, for example.

FIG. 3B, as referenced above, shows another exemplary embodiment of a lead 100 coupled to a biomedical device 300. As shown in FIG. 3B, lead 100 is coupled to a biomedical device 300 at or near the proximal end 104 of lead 100, and lead 100 comprises a distal component 116 coupled to lead 100 at or near the distal end 106 of lead 100. Such an embodiment has similarities to the lead 100 embodiment shown in FIG. 3B, noting that in the embodiment shown in FIG. 3B, biomedical device 300 is the effective proximal component. An external component 310, as shown in FIG. 3B, may be coupled to distal component 116, so that a signal transferred through lead 100 can be carried through distal component 116 to external component 310. In various embodiments, distal component 116 may be a connector, and external component 310 may be an electrode (which may be referred to as an “exposed electrode”), a connector, and/or a closure device, for example.

In operation, electrical signals may be transmitted through lead 100 through electrically conductive composition 200 from biomedical device 300 to electrode distal component 116 (such as an electrode, for example), thereby causing therapeutic stimulation of a remote target tissue adjacent to distal component 116. Contrary to prior applications utilizing solid metals, helically wound metal ribbons, or other embodiments that are subject to fracturing, various leads 100 of the present disclosure utilize an electrically conductive composition 200 having a melting point at or below 98° F. (or otherwise being a liquid or a gel at or below 98° F.) such that the conductor is a liquid and thereby significantly less susceptible to fracture. According to at least one embodiment, electrically conductive composition 200 is contained within first interior lumen 112 of lead 100, which effectively maintains a continuous liquid conduction path from biomedical device 300 to distal component 116 (such as an electrode, for example). According to at least one embodiment, first interior lumen 112 (or one or more other lumens of the present disclosure) is filled with electrically conductive composition 200 under vacuum or under atmospheric pressure. In another embodiment, electrically conductive composition 200 is maintained under vacuum or under atmospheric pressure. In such embodiments, leads 100 are prepared in a way to prevent gaseous pockets (bubbles) from forming within electrically conductive composition 200 which can disrupt the ability of lead 100 to conduct a signal from biomedical device 300 to distal component 116, and vice versa.

Further, according to at least one embodiment, electrically conductive composition 200 may comprise a conductive polymer, an electrically conductive liquid, metal, alloy, etc. as described herein, with an appropriate conductivity and resistivity to allow conduction of an appropriate electric current over the required length of biomedical device lead 100 and to administer a therapeutic current to a target tissue. By way of nonlimiting examples, alloys and metals that may be utilized as electrically conductive compositions 200 may include, but are not limited to, gallium, its alloys (such as gallium-indium alloys, for example), Galinstan, a conductive polymer, an electrically conductive liquid, an electrically conductive gel, or combinations thereof. In various embodiments, electrically conductive composition 200 is biologically-compatible (or non-toxic), so that in the unlikely event of lead 100 breakage, electrically conductive composition 200 will not have a detrimental effect to the patient. It will be appreciated that the resistivity of gallium or its alloys may exceed that of common biomedical device wire materials such as MP35N, 35N LT, or similar alloys, platinum or iridium alloys, copper, gold, or platinum. However, liquid metal alloy-based leads may exhibit a larger cross-sectional areas than large gauge, helically wound solid metal conductors often utilized to reduce fracturing, thereby decreasing the overall resistance of the lead, and allowing electric currents to be transmitted through a lower resistance path than that utilized in conventional implantable biomedical leads. In addition, the use of a continuous liquid metal conductor within lumen 112 does not increase the overall length of conductors as required by helically winding smaller conductor wires or ribbons, further reducing the overall resistance that necessarily accompanies the lengthening of a conductor.

According to at least one embodiment, an electrically conductive composition suspended in liquid or gel form may further comprise the electrically conductive composition 200, and any such electrically conductive liquid wire less than 20 ohms of resistance, for example, may be utilized as a conductor for low current applications, e.g., implantable or external cardiac pacemakers, while electrically conductive solutions having no more than 1 ohm, and optionally no more than 0.5 ohms (500 milliohms) of resistivity may be utilized for high current applications, e.g., an implantable cardioverter defibrillator. For example, low current applications, such as pacing stimulation, do not require significant current, and those currents may be transferred by solutions having a relatively high resistance, depending upon the cross-section of the lead required as well as the overall length. Medium current applications such as neural stimulation have intermediate requirements.

Various leads 100 of the present disclosure may be used with implantable or external cardiac pacemakers for treatments of cardiac arrhythmias, cardioverter/defibrillators for delivering cardio version/defibrillation shocks to a distal electrode, as a neural stimulator lead, or for various other implantable or external electrical stimulator/measurement device lead applications, such as applications in connection with electrocardiogram (EKG or ECG) devices and electroencephalogram (EEG) devices, for example. Each said component may be viewed as an exemplary biomedical device 300 of the present disclosure. Accordingly, and as referenced generally herein, various leads 100 of the present disclosure can be used with various sensing and stimulating biomedical devices 300, conducting electrical signals to and from said devices 300.

The cumulative effects of repetitive movement can cause fractures (breaks) to form in the electrically conductive materials used in traditional implantable leads. Such fractures may ultimately compromise or completely inhibit the ability of the lead to either transmit electrical signals to the target tissue, or to sense (detect) intrinsic electrical activity. Exemplary leads 100 of the present disclosure overcome this problem, as the electrically conductive composition 200 is not susceptible to fracture.

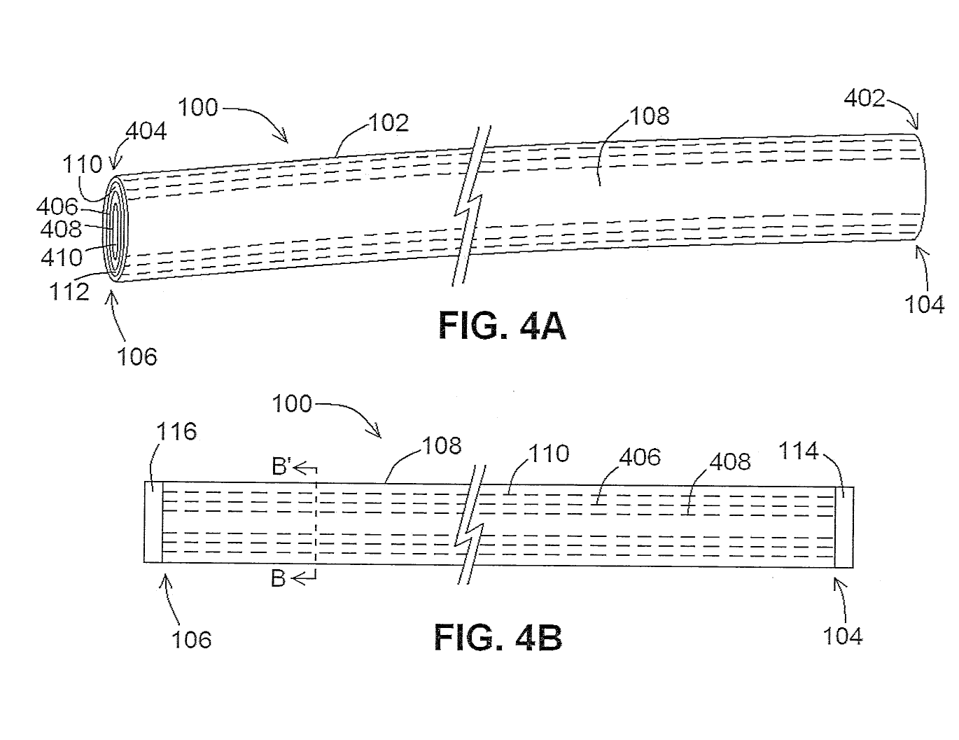

At least another exemplary embodiment of a lead 100 of the present disclosure is shown in FIGS. 4A and 4B. As shown in FIG. 4A, lead 100 comprises a lead body 102 having a proximal end 104 and a distal end 106, and defining a first outer surface 108 and a first inner surface 110. In the lead 100 embodiment shown in FIG. 4A, lead 100 effectively comprises a “double sheath,” as lead 100 has a second sheath, identified generally as interior tube 400, positioned within first interior lumen 112 defined by lead body 102. Interior tube 400, as shown in FIG. 4A, has a proximal end 402 and a distal end 404, and itself defines a second outer surface 406 and a second inner surface 408, with interior tube 400 further defining a second interior lumen 410 therein (surrounded by second inner surface 408).

FIG. 4B shows a side view of an exemplary lead 100 of the present disclosure having a double sheath. As shown in FIG. 4B, the dotted lines represent the internal walls of lead 100, namely first outer surface 108, first inner surface 110, second outer surface 406, and second inner surface 408. As shown in FIG. 4B, lead 100 may comprise a proximal component 114 at or near the proximal end 104 of lead body 102, and may comprise a distal component 116 at or near the distal end 106 of lead body 102.

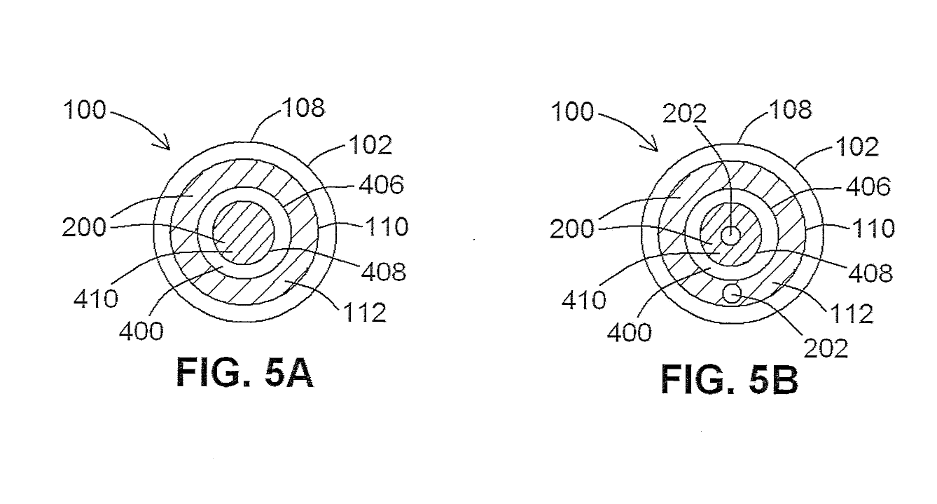

FIG. 5A shows cross-sectional views of lead 100 along section B-B′ as shown in FIG. 4B. As shown in FIG. 5A, operable leads 100 of the present disclosure comprise an electrically conductive composition 200 present within either first interior lumen 112 and/or second interior lumen 410 as may be the case with one or more embodiments of leads 100 of the present disclosure. Electrically conductive composition 200, as shown in FIG. 5A, is depicted as a series of diagonal lines FIG. 5B shows a cross-sectional view of another embodiment of a lead 100 of the present disclosure, comprising a conductor wire 202 positioned within first interior lumen 112 and another conductor wire 202 positioned within second interior lumen 410. In various embodiments of leads 100 of the present disclosure, leads 100 may have one or more conductor wires 202 positioned within first interior lumen 112, second interior lumen 410, and/or embedded within lead body 102 and/or tube 400 itself. Said conductor wires 202, in various embodiments, could coil within first interior lumen 112 around second interior lumen 410.

According to at least one embodiment of a lead 100 of the present disclosure, such as shown in FIGS. 5A and 5B, first interior lumen 112 and/or second interior lumen 410 may be filled with, or otherwise be in fluid contact with, electrically conductive composition 200. Lead body 102 and/or tube 400 may themselves comprise a polymer or other material compatible with the selected electrically conductive composition 200, or portions thereof may be covered with a polymer or other material compatible with the electrically conductive composition 200. For example, an effective outer sheath of lead 100 may substantially encapsulate an effective interior sheath, with one or both comprising or covered with a polymer or other material compatible with the electrically conductive composition 200. As electrically conductive compositions 200 of the present disclosure may themselves be corrosive or caustic to certain other materials, creation of such a dual-sheath, as shown in FIGS. 4A-5B, allows for biocompatibility along with longevity of lead 100.

Various embodiments of leads 100 of the present disclosure may be unipolar, namely that lead 100 has one physical connection to another device, such as a pacemaker, for example. In such an embodiment, such as the lead 100 embodiment shown in FIG. 2A, lead 100 may be used to connect a device to a heart, whereby the return path of the circuit flows through the body. In such an embodiment, an electrical signal can be transmitted to or from the heart, and the patient's body completes the circuit. Such an embodiment may be used for a sensing application, for example.

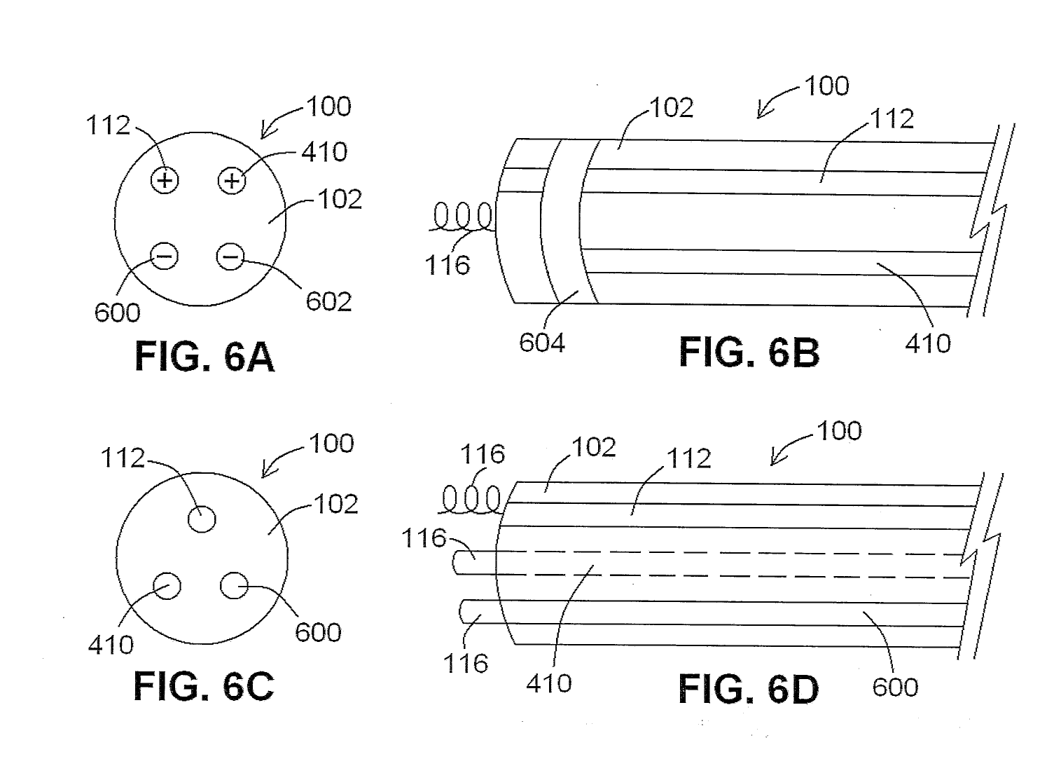

In other embodiments, leads 100 of the present disclosure may be bipolar, meaning that lead 100 has two physical connections to another device, such as a pacemaker, forming a cathode and an anode, for example. In such an embodiment, such as the lead 100 embodiment shown in FIG. 5A, lead 100 may be used to connect a device to a heart, whereby the return path of the circuit flows through the lead 100. In such an embodiment, an electrical circuit can be completed by sending a signal to the heart through one conductor and sending a return signal from the heart using a second conductor. Furthermore, various leads 100 of the present disclosure may be multipolar, which is defined herein as having three or more conductor paths. In such an embodiment, such as the leads 100 shown in FIGS. 6A and 6C, signals can be sent to and from a heart using lead 100, and such signals, for example, can be redundant using two or more lead conductors. Such an embodiment may be used for a sensing application and a pacing application, for example, given the two physical conductors within said lead 100.

As shown in the cross-sectional view of exemplary lead 100 shown in FIG. 6A, lead 100 comprises a lead body 102 having four channels/lumens. In at least one embodiment, said lumens comprise first interior lumen 112, second interior lumen 410, third interior lumen 600, and fourth interior lumen 602, each defined within lead body 102. One or more of said lumens 112, 410, 600, 602 may be filled with an electrically conductive composition 200 as described herein, and depending on configuration of lead 100, signals may be transmitted in a first direction (indicated by the (+) shown in FIG. 6A) and in a second direction (indicated by the (−) shown in FIG. 6A). The two (+) identifiers and the two (−) identifiers in FIG. 6A indicate either redundant conductor paths with signals flowing in opposite directions, or two separate conductor paths with signals flowing in one direction and two separate conductor paths with signals flowing in an opposite direction.

FIG. 6B shows a side view of at least a portion of lead 100 of the present disclosure that could be used as a bipolar lead 100. As shown in FIG. 6B, lead 100 comprises a lead body, with two lumens (selected from 112, 410, 600, and 602, with 112 and 410 selected in the present example). Distal component 116, shown in FIG. 6B, may comprise a coil configured to permanently or removably engage a targeted tissue. As shown in FIG. 6B, a cuff 604 may be positioned at least partially circumferentially around lead 100, said cuff 604 configured, in at least one embodiment, to compress at least a portion of lead 100 so that a distal component 116 remains affixed thereto. Said embodiment, as well as other bipolar or multi-lumen (multipolar) leads 100 of the present disclosure, may be referred to herein as having redundant conductors.

FIGS. 6C and 6D show additional embodiments of leads 100 of the present disclosure that could also be used as bipolar leads 100. FIG. 6C shows a cross-sectional view of lead 100, comprising a lead body 102 and three lumens (selected from 112, 410, 600, and 602, with 112, 410, and 600 selected in the present example). FIG. 6D shows a side view of at least a portion of lead 100 shown in FIG. 6C, wherein lumen 112 is configured as a stylet lumen, and wherein lumens 410 and 600 are filled with an electrically conductive composition 200 to conduct signals to and from the respective ends 104, 106 of lead 100. Alternatively, and in various embodiments, lumen 410 could be filled with a solid conductor, and lumen 112 could be filled with an electrically conductive composition 200. Stylet lumens of various embodiments of leads 100 of the present disclosure may be configured to allow a stylet to be positioned therethrough, whereby the stylet can be used to deliver lead 100 (or portions thereof) to a desired location within a body, for example, and/or used to engage portions of lead 100 to facilitate engagement of lead 100 components (such as distal component 116, for example), at a desired location within a body and/or to engage an electrode, for example. Lead 100, as shown in FIG. 6D, may have three distal components 116 comprising coils and/or electrodes as shown therein.

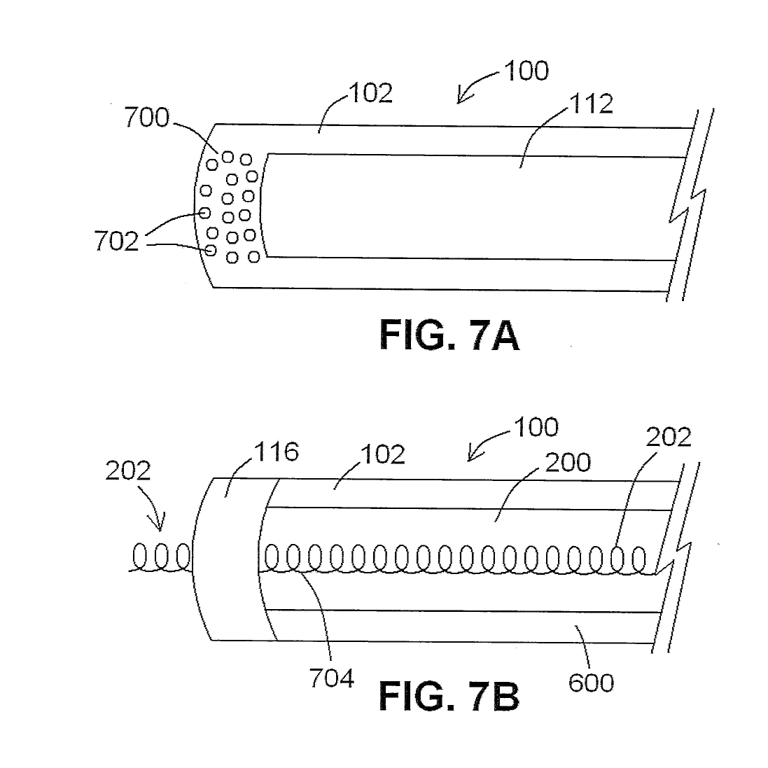

FIGS. 7A and 7B show additional embodiments of exemplary leads 100 of the present disclosure. FIG. 7A shows at least part of an exemplary lead 100 of the present disclosure having a doped portion 700 at or near distal end 106 of lead 100. Doped portion 700, in various embodiments, comprises a conductive material 702 embedded therein, so that a signal transmitted through an electrically conductive composition 200 within lead body 102 may further be transmitted through doped portion 700 and optionally any distal component 116 adjacent thereto. In various embodiments, conductive material 702 is comprised of, but not limited to, stainless steel, platinum, titanium, silver, tantalum, MP35N (an exemplary nickel-cobalt base alloy), 35N LT (another exemplary nickel-cobalt base alloy), carbon black, carbon fiber, nanofiber tubes, polyaniline, polypyrrole, polythiophene, polyethylenedioxythiophene, poly(p-phenylene vinylene), and/or other materials useful to maintain electrical conductivity.

FIG. 7B shows an exemplary embodiment of a lead 100 of the present disclosure having a conductor wire 202 positioned therein, similar to the embodiment of lead 100 shown in FIGS. 2B and 2C. However, the embodiment of lead 100 shown in FIG. 7B has a coiled conductor wire 202 positioned therein, wherein said conductor wire 202 is coated by insulated coating 704. In such an embodiment, for example, a first signal may be transmitted through an electrically conductive composition 200 within lead 100, and a second signal may be transmitted through conductor wire 202. Conversely, electrically conductive composition 200 and conductor wire 202 could both be used to transmit the same (or redundant) signal through lead 100.

In addition, and as shown in FIG. 7B, lead 100 may comprise a distal component 116, and in at least one embodiment, conductor wire 202 may be positioned through distal component 116 so that a non-coated portion of conductor wire 202 is exposed at a distal end 106 of lead 100. In such an embodiment, a first signal may travel through electrically conductive composition 200 to distal component 116 (a sensing electrode, for example), and a second signal may travel through conductor wire 202 to a targeted tissue adjacent to the exposed portion of conductor wire 202 of lead 100.

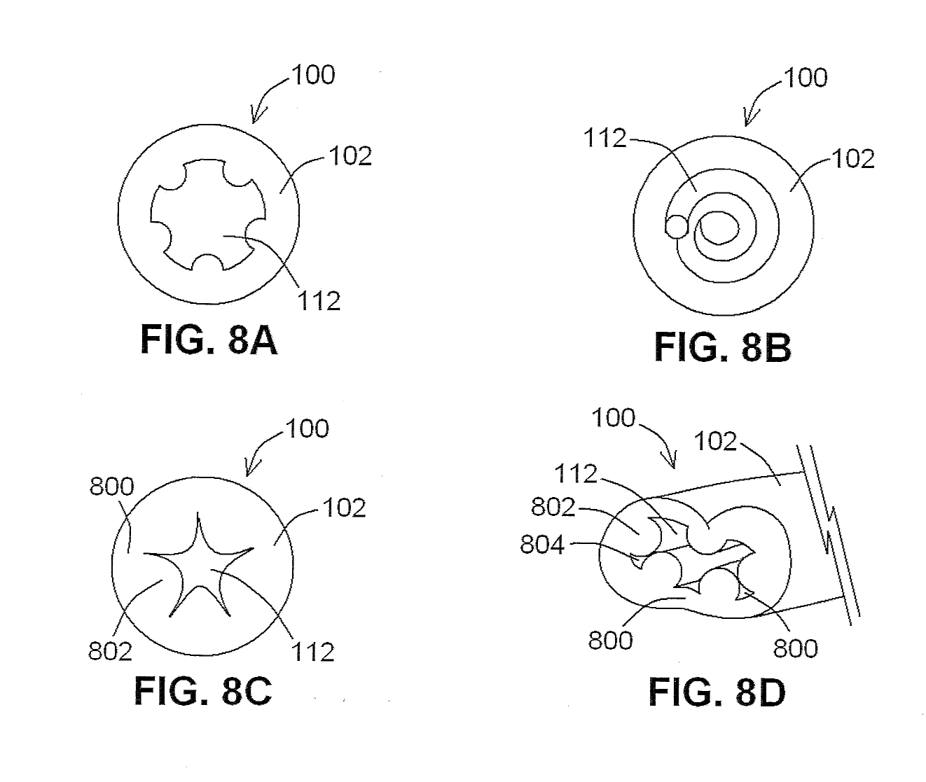

FIGS. 8A, 8B, and 8C show various configurations of first interior lumens 112 of exemplary leads 100 of the present disclosure. As shown in the cross-sectional view of an exemplary lead 100 of the present disclosure shown in FIG. 8A, lead 100 defined a first interior lumen 112 having a modified pentagonal cross-section. Such an embodiment, for example, could allow for relatively thicker portions of lead body 102 to exist along with relatively thinner portions of lead body 102 so that, for example, lead 100 is not only structurally sound but also flexible as needed/desired for a particular application, and so that lead 100 may maintain active conductance in the event of compression. In addition, and in such an embodiment, various lumens 410, 600, 602, and others can be more readily defined within the relatively thicker portions of lead body 102, so that such lumens can include a conductor or provide stylet access, for example. Lead bodies 102 of the present disclosure are not limited to the circular and modified pentagonal cross-sections as described and shown herein, as various other embodiments, such as hexagonal, septagonal, octagonal, and others may be used depending on the desired application.

The cross-sectional view of the exemplary lead 100 of the present disclosure shown in FIG. 8B shows a lead body 102 having a coiled first interior lumen 112 defined therethrough. In such an embodiment, for example, a first interior lumen 112 defined within lead body 102 can be filled with an electrically conductive composition 200, so that for a given length of lead 100 and diameter of first interior lumen 112, more electrically conductive composition 200 would be present in an embodiment of lead 100 having a coiled first interior lumen 112 instead of a straight first interior lumen 112.

FIG. 8C shows a cross-sectional view of another exemplary embodiment of a lead 100 of the present disclosure. As shown in FIG. 8C, lead 100 comprises a lead body 102 having relatively thick and relatively thin portions which define an irregular star-shaped lumen 112. Such an embodiment may provide additional strength to lead body 102, may provide the physical cross-sectional area to add one or more additional lumens within lead body 102, and so that lead 100 may maintain active conductance in the event of compression. As shown therein, the irregularly-shaped lumen 112, defined by lead body 102, results in lead body 102 having one or more relatively thinner portions 800 and one or more relatively thicker portions 802.

FIG. 8D shows a perspective cross-sectional view of an exemplary lead 100 of the present disclosure that has been compressed. As shown in FIG. 8D, lead body 102 is defined the same as or somewhat similar to lead 100 shown in FIG. 8A, and compression thereof forms localized conductance pockets 804 that, if first interior lumen 112 filled with an electrically conductive composition 200, conductance can be maintained through said lead 100 in the event of compression/bending as conductance pockets 804 would not fully close. As shown in FIG. 8D, a conductance pockets 804 may be formed when two relatively thicker portions 802 of lead body 102 engage one another.

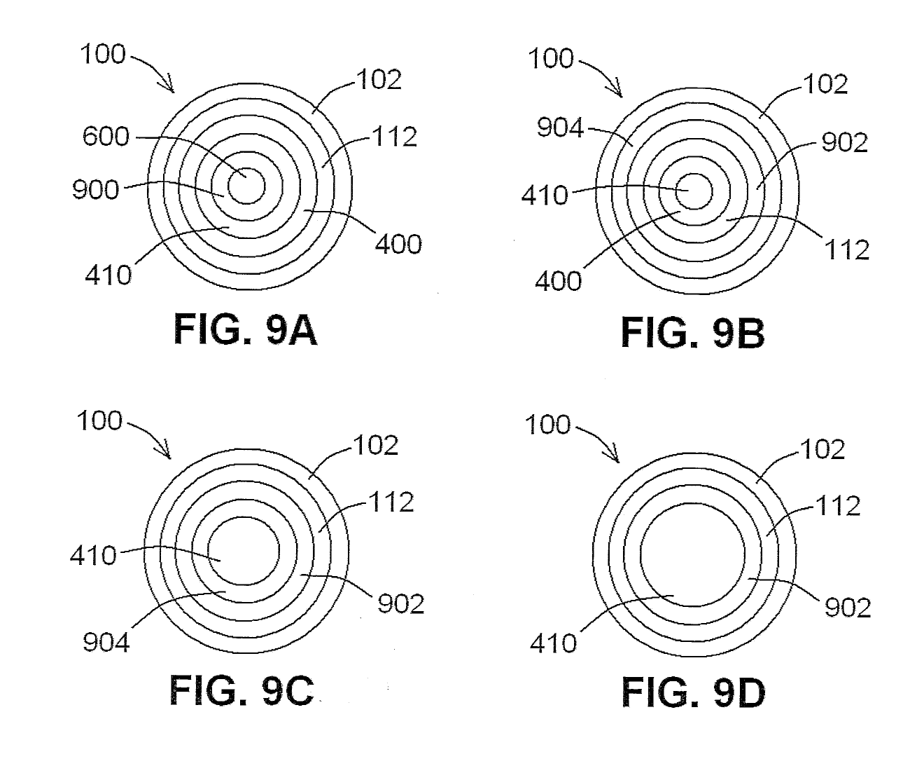

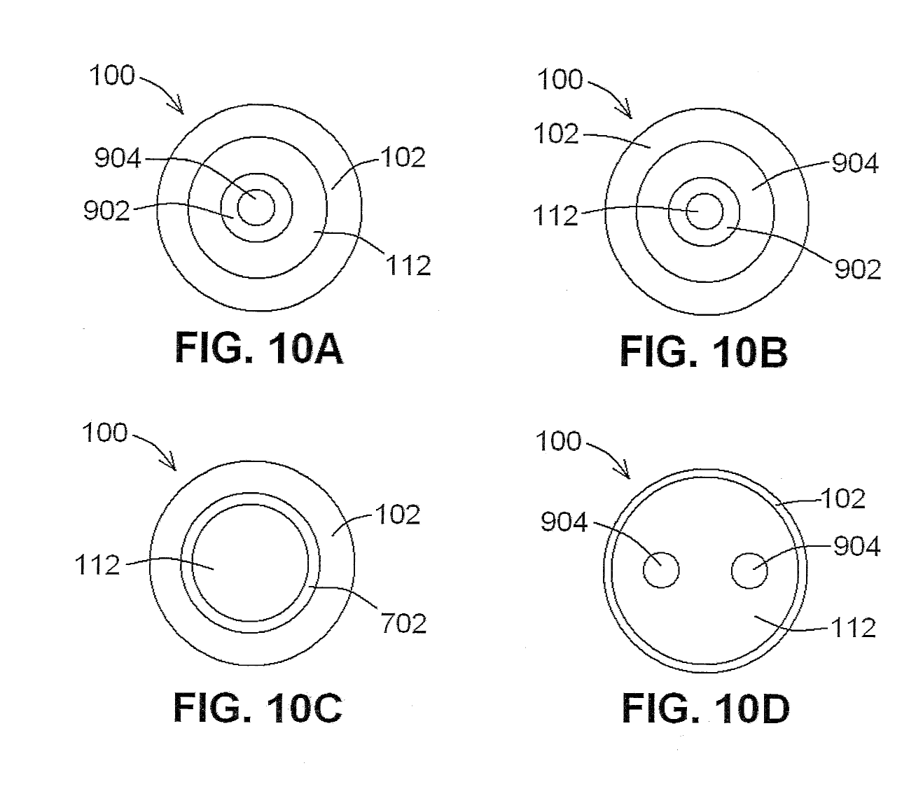

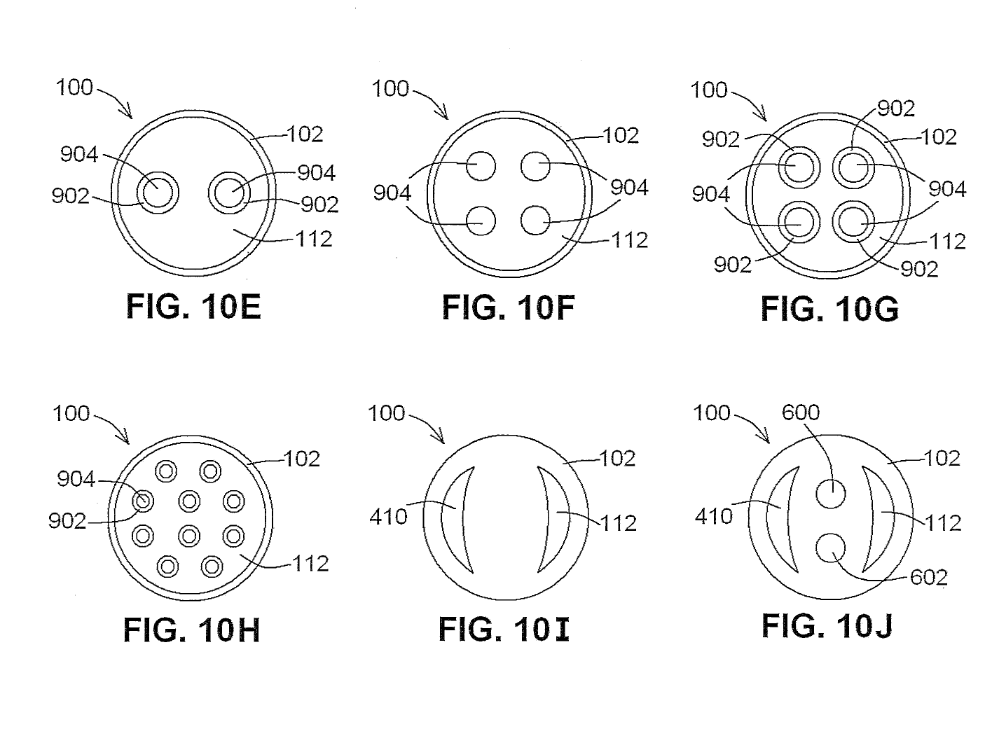

Various cross-sectional views of exemplary leads 100 of the present disclosure are shown in FIGS. 9A-10C. As shown in FIG. 9A, an exemplary lead 100 comprises the following layers/elements as viewed from the outside of lead 100 to the relative center of lead 100: a lead body 102, a first interior lumen 112, a first interior tube 400 (the wall of said tube 400), a second interior lumen 410 (defined within first interior tube 400), a second interior tube 900 (the wall of said tube 900), and a third interior lumen 600. In such an embodiment, first interior tube 400 and second interior tube 900 may either or both be viewed as insulators, effectively insulating portions of lead 100 from one another. In addition, and in such an embodiment, first interior lumen 112 and second interior lumen 410 may be filled with an electrically conductive composition 200, and third interior lumen 600 may serve as a stylet lumen, for example, at the relative middle/center of lead 100.

FIG. 9B shows a cross-section of an exemplary lead 100 of the present disclosure having the following layers/elements as viewed from the outside of lead 100 to the relative center of lead 100: a lead body 102, a solid metal component/layer 904, a non-conductive insulator 902, a first interior lumen 112, a first interior tube 400 (the wall of said tube 400), and a second interior lumen 410 (defined within first interior tube 400). In such an embodiment, first interior lumen 112 may be filled with an electrically conductive composition 200, and second interior lumen 410 may serve as a stylet lumen, for example, at the relative middle/center of lead 100.

FIG. 9C shows a cross-section of another exemplary lead 100 of the present disclosure having the following layers/elements as viewed from the outside of lead 100 to the relative center of lead 100: a lead body 102, a first interior lumen 112, an insulator 902 (or a first interior tube 400 (the wall of said tube 400)), a solid metal component/layer 904, and a second interior lumen 410 (defined within solid metal component/layer 904). In such an embodiment, and similar to the embodiment of lead 100 shown in FIG. 9B, first interior lumen 112 may be filled with an electrically conductive composition 200, and second interior lumen 410 may serve as a stylet lumen, for example, at the relative middle/center of lead 100.

FIG. 9D shows a cross-section of an exemplary lead 100 of the present disclosure having two lumens 112, 410 for placement of an electrically conductive composition 200 therein. As shown in FIG. 9D, lead 100 has the following layers/elements as viewed from the outside of lead 100 to the relative center of lead 100: a lead body 102, a first interior lumen 112, an insulator 902 (or a first interior tube 400 (the wall of said tube 400)), and a second interior lumen 410 (defined within insulator 902 or first interior tube 400). In such an embodiment, first interior lumen 112 and second interior lumen 410 may be filled with an electrically conductive composition 200 so that such an embodiment of lead 100 may operate as a bipolar lead if desired.

Lead 100, as shown in FIG. 9D and as otherwise described and/or shown herein in various other embodiments of leads 100, may be designed and/or used as bipolar leads 100 as generally referenced herein. For example, and as shown in FIG. 9D, lead 100 comprises both a cathode (a negative terminal, typically active) and an anode (a positive terminal, typically inactive or return). According to at least one embodiment, the creation of a cathode and anode is accomplished by infusing a liquid metal into a sheath of biocompatible, electrically insulating material, forming the active electrode. The inactive electrode, typically the anode, is optionally encapsulated either concentrically or co-longitudinally with the active electrode. This is performed by, for example, placing an insulator 902 (or a first interior tube 400) within tube body 102, and filling the first interior lumen 112 the second interior lumen 400 (defined by either insulator 902 or first interior tube 400) with an electrically conductive composition 200, as shown in FIG. 9D. In such a configuration, the active electrode (defined by electrically conductive composition 200 within the first interior lumen 112) is surrounded by a larger diameter, electrically insulating, biocompatible sheath (namely, tube body 102). The space between the inner and outer sheaths (namely lead body 102 and insulator 902/first interior tube 400) is filled with the liquid metal electrically conductive composition 200, effectively forming a coaxial liquid wire as shown in FIG. 9D. In addition, said lead 100 embodiment, as well as various other lead embodiments 100 having multiple poles/lumens, may be less susceptible to pinching/kinks when in use, which may cause potential leakage of electrically conductive composition 200 and/or a loss of conductivity within lead 100.

In an optional co-longitudinal configuration, two columns of liquid metal will be encapsulated in biocompatible, electrically insulating polymer sheaths, as shown in FIG. 6B, for example. The liquid columns, namely the first interior lumen 112 and the second interior lumen 410 filled with electrically conductive composition 200, are optionally placed in a larger diameter biocompatible polymer sheath (namely lead body 102), with the columns longitudinal axes parallel to each other. In at least one embodiment, and to minimize capacitive coupling, the liquid columns may be twisted into a pair. Additionally, in such a configuration, the columns are optionally surrounded by an additional insulator 902, and an additional port, namely a third interior lumen 600, included therein to facilitate lead placement via a semi-rigid stylus (a stylet), as shown in the exemplary embodiment shown in FIG. 6C.

Another exemplary lead 100 of the present disclosure is shown in FIG. 10A. As shown in the cross-sectional view of lead 100 shown in FIG. 10A, lead 100 has the following layers/elements as viewed from the outside of lead 100 to the relative center of lead 100: a lead body 102, a first interior lumen 112, an insulator 902 (or a first interior tube 400 (the wall of said tube 400)), and a solid metal component/layer 904 at the relative center of lead 100, said solid metal component/layer 904 also being referred to as a conductor wire 202, as shown in FIG. 2B and described herein. An electrically conductive composition 200 may be placed within first interior lumen 102 so that such an embodiment of lead 100 may also operate as a bipolar lead if desired.

In such an embodiment, for example, lead 100 uses potentially redundant current paths, namely a first path using an electrically conductive composition 200 and a second path using a solid metal component/layer 904. In such an embodiment, the electrically conductive composition 200 is used in conjunction with conventional implanted lead materials. The conventional solid lead assembly is not altered; liquid metal (the electrically conductive composition 200) is instead infused and in direct contact with the solid metal conductor (solid metal component/layer 904). Both materials are encapsulated by an insulating, biocompatible polymer, namely lead body 102 and insulator 902, for example, as shown in FIG. 10A. The liquid metal conductor thereby acts as an alternate conduit, providing a low impedance path for current to be transmitted in the event of solid conductor fracture.

FIG. 10B shows a cross-section of yet another exemplary lead 100 of the present disclosure As shown in FIG. 10B, lead 100 has the following layers/elements as viewed from the outside of lead 100 to the relative center of lead 100: a lead body 102, a solid metal component/layer 904, an insulator 902 (or a first interior tube 400 (the wall of said tube 400)), and a first interior lumen 112 for containing an electrically conductive composition 200.

FIG. 10C shows a cross-section of an additional embodiment of a lead 100 of the present disclosure. In such an embodiment, first interior lumen 112 (and/or other lumens 410, 600, 602, or others) may be non-conductive or clad with a thin coating of a conductive material (such as a conductive material 702 used with doped ends of various leads 100 or metals used in solid metal component layers 904 of the present disclosure), thereby allowing conduction of electrical signals throughout the surface of said lumen 112 (or other lumens) and be in contact with an electrically conductive composition 200.

FIGS. 10D and 10E show cross-sectional views of additional embodiments of leads 100 of the present disclosure. As shown in FIG. 10D, lead 100 comprises two solid metal components/layers 904 therein, so that if first interior lumen 112 is filled with an electrically conductive composition 200, lead 100 would carry a redundant signal through electrically conductive composition 200 and solid metal components/layers 904. If the solid metal components/layers 904 were coated with an insulator 902, as shown in FIG. 10E, separate signals could be transmitted through electrically conductive composition 200 and solid metal components/layers 904. FIGS. 10F and 10G show exemplary lead 100 embodiments with four solid metal components/layers 904, without insulators 902 (FIG. 10F) and with insulators 902 (as shown in FIG. 10G). FIG. 10H shows an exemplary lead 100 embodiment with eight solid metal components/layers 904 coated with insulators 902. Various other embodiments of leads 100 having any number of solid metal components/layers 904 with and without coatings 902 are also within the scope of the present disclosure.

FIGS. 10I and 10J show cross-sections of exemplary embodiments of leads 100 of the present disclosure with crescent-shaped lumens. As shown in FIG. 10I, lead body 102 defines a first interior lumen 112 and a second interior lumen 410, whereby said lumens 112, 410 have a cross-sectional shape with a relatively wide portion terminating at both ends to a point. Such an embodiment, for example, can be readily compressed, providing desired strain relief in various embodiments. Accordingly, lumens having such a cross-sectional shape may be referred to herein as “strain relief lumens,” noting that other cross-sectional shapes of lumens may also effectively function as strain relief lumens, such as round lumens. However, crescent-shaped lumens, or lumens having at least one and perhaps two points at opposing ends and a relatively wide portion therebetween are more readily susceptible to compression. FIG. 10J shows an exemplary lead 100 embodiment with four lumens, whereby lumens 112 and 410 are strain relief lumens, and 600 and 602 are regular lumens. One or more such lumens 112, 400, 600, 602 may be filled with an electrically conductive composition 200 as referenced within the present disclosure, whereby electrically conductive composition 200 confirms to the various shapes of said lumens. In addition to the foregoing, the overall flexibility of various leads 100 of the present disclosure, including the use of a electrically conductive composition 200 that is capable of deforming and confirming to lumen(s) of said leads 100, may obviate the need for additional compression lumens.

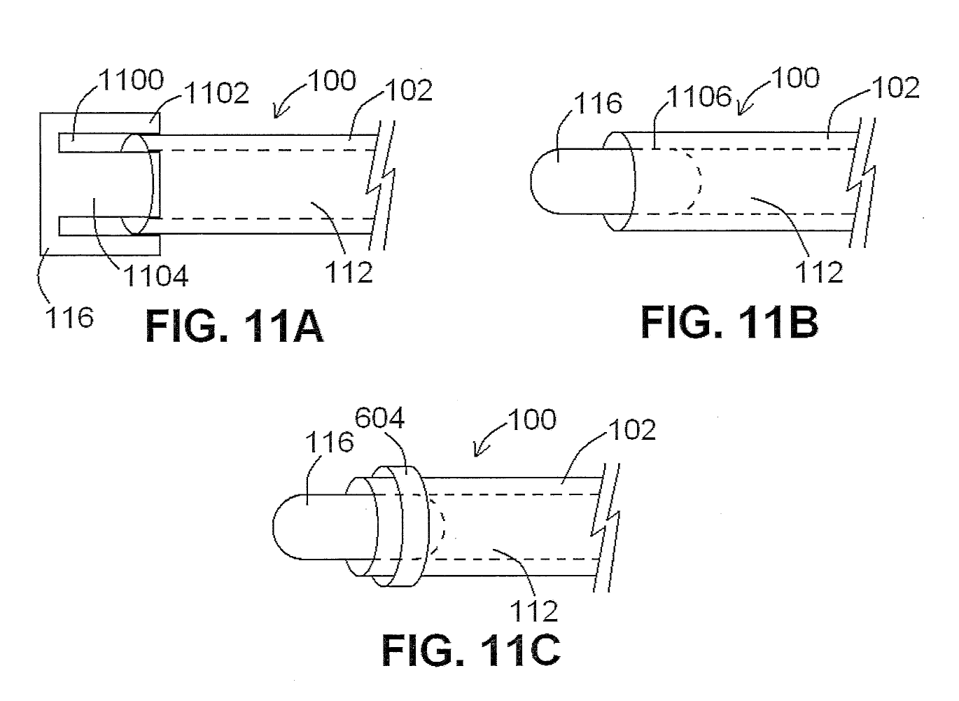

Various leads 100 of the present disclosure may comprise distal components 116 of differing configurations other than those otherwise described herein. For example, and as shown in the cross-sectional view of a portion of an exemplary lead 100 of the present disclosure shown in FIG. 11A, an exemplary distal component 116 of the present disclosure may comprise a distal component 116 having a circumferential groove 1100 defined therein, so that an outer portion 1102 of distal component 116 may positioned on the relative outside of lead body 102, and so that an inner portion 1104 of distal component 116 may be positioned in the relative inside of lead body 102. In such an embodiment, lead body 102 itself is sized and shaped to fit within circumferential groove 1100 of distal component 116, so that distal component 116 effectively seals the distal end 106 of lead body 102.

FIGS. 11B and 11C show exemplary distal components 116 of the present disclosure configured as pins. As shown in FIG. 11B, distal component 116 is pin-shaped, so that distal component has an outer perimeter sized and shaped to fit within a first interior lumen 112 of a lead body 102, effectively sealing the distal end of lead body 102. Distal component 116, in various embodiments, may have an adhesive 1106 positioned thereon and/or an adhesive 1106 may be positioned within first interior lumen 112 so that distal component 116 may more effectively seal the distal end 106 of lead body 102. As shown in FIG. 11C, a cuff 604 may also be positioned around lead body 102, whereby cuff 604 exerts pressure against lead body 102 (by way of the configuration of cuff 604 and/or by crimping cuff 604 against lead body 102) to facilitate sealing of the distal end 106 of lead body 102 along with distal component 116. Such compression may be a localized crimping, or may involve swaging, namely a full concentric pinch to fully concentrically compress cuff 604 around lead body 102.

In various other embodiments, various proximal components 114 of the present disclosure may be configured as the various distal components 116 shown in FIGS. 11A-11C are configured.

Embodiments of leads 100 without metals as compared to embodiments of leads 100 comprising metal(s) (such as a conductive material 702 or a metal used in a solid metal component layer 904 of the present disclosure) may have different applications. For example, various embodiments of leads 100 with and without metals may have different conductivity capabilities, different relative lives, different perforation capabilities (as less rigid leads 100 are less capable of tissue perforation as compared to more rigid leads 100), potential dislodgment rates, pliabilities, and the ability to produce leads 100 of various sizes.

As referenced herein, various leads 100 of the present disclosure solve for three main lead failures, namely conductor failure, insulator failure, and perforation failure. Conductor failure is solved by way of using an electrically conductive composition 200 with or without a redundant solid metal conductor, in various embodiments. Insulator failure is solved by designing leads 100 whereby the conductive portions are properly matched to the insulative portions, namely lead body 102 and other insulators. By filling various lumens of leads 100 with an electrically conductive composition 200 that conforms to the lumen shape(s), leads 100 can maintain conductivity even in the event of significant bending or crimping of said leads 100. Perforation failure is also solved as various leads 100 of the present disclosure can have superior flexibility, allowing for additional bending as needed, as well as relatively larger cross-sectional areas as the electrically conductive composition 200, as noted below, has excellent impedance properties so that less power is needed to conduct a signal therethrough as compared to traditional leads as various leads 100 of the present disclosure have a lower resistance as compared to traditional leads. Such physical embodiments decrease the potential for incidences of myocardial perforation, as perforation rates increase when leads of relatively small cross-sectional areas are used. Phrased another way, relatively thick and relatively flexible leads are less likely to cause myocardial perforation as compared to relatively thin and relatively stiff leads.

In various embodiments of leads 100 of the present disclosure, leads 100 are capable of transmitting an electrical signal through electrically conductive composition 200 at a lower resistance than a traditional lead having equivalent dimensions but using only a solid conductor. In any number of lead 100 embodiments, lead 100 is capable of transmitting an electrical signal through electrically conductive composition 200 resulting in less signal energy loss in lead impedance through electrically conductive composition 200 as compared to signal energy losses in lead impedance through a solid conductor of a traditional lead. For example, ICD leads typically have a resistance of 2Ω-8Ω, and load impedance at the tissue-electrode interface of 50Ω. The comparable impedances between the load and lead can result in significant energy drops across the lead, reducing overall efficiency of the ICD. For example, an ICD delivering electrical stimuli through a 6.8Ω lead resistance and a 50Ω termination resistance will lose at least 12% of the pulse energy due to conduction (i2R) losses through the lead. Using an embodiment of a lead 100 of the present disclosure with a 120 mΩ (bipolar) lead resistance, for example, it is predicted that 0.2% of the signal energy would be lost in the lead impedance, significantly increasing the energy efficiency of the ICD.

In various embodiments of leads 100 of the present disclosure, lead body 102 comprises a material capable of flexion so that repeated bending of lead body 102 does not result in loss of conductance through electrically conductive material 200. In various embodiments, electrically conductive composition 200 is sufficiently pliable so to reduce a likelihood of fracture of lead body 102 at a location where at least a portion of electrically conductive composition 200 contacts at least a portion of lead body 102. In various embodiments, lead body 102 is sufficiently flexible and electrically conductive composition 200 is sufficiently pliable so to reduce a likelihood of tissue perforation within a patient's body when lead 100 is positioned within the patient's body and coupled to the tissue as compared to a traditional lead having a solid conductor.

In various embodiments of leads 100 of the present disclosure, leads 100 may comprise a metallic electrically conductive composition 200, such as gallium, a gallium-indium alloy, Galinstan, its/their alloys, or combinations thereof, as well as a non-metallic electrically conductive composition 200, such as a conductive polymer, an electrically conductive liquid, an electrically conductive gel, or combinations thereof.

### EXAMPLE 1

According to at least one exemplary embodiment of a lead 100 of the present disclosure, a unipolar liquid metal lead 100 was developed by infusing liquid gallium (Ga 4N, Recapture Metals) into a polyurethane tube. The tube dimensions were comparable to those of a conventional cardiac pacing lead, having a length of 610 mm and an inner diameter of 1.7 mm. The tube was filled with Ga such that the lumen appeared free of any voids and terminated on each end with segments of 22 AWG solid core wire. The resultant liquid lead 100 was connected to a bipolar amplifier (Kepco BOP100-2M) operating in a constant current mode. DC currents of varying amplitudes were driven through lead 100, and the voltage across the lead was measured using an oscilloscope (Agilent 54621A). Using Ohm's law, it was determined that the resistance of lead 100 was 61 mΩ, which is consistent with the predicted resistance of a column of Ga having the same dimensions.

The time-varying characteristics of the liquid metal lead 100 were assessed by connecting lead 100 in series with a load resistance (R_{L}=10.5 ). A sinusoidal voltage was applied across lead 100 and R_{L} and the voltage across the liquid lead (V_{Ga}) was measured. The frequency of the sinusoidal voltage was increased from DC to 10 kHz, and the voltage of lead 100 was examined to determine whether lead 100 impedance changed as function of frequency. V_{Ga} was approximately the same at all frequencies, indicating that lead 100 was a primarily resistive element, as there was no discernible phase shift between the input voltage and the voltage across lead 100 at any frequency of interest. This is beneficial because lead 100 will behave as a low impedance resistor, and therefore signals transmitted through lead 100 will not be distorted in amplitude or phase.

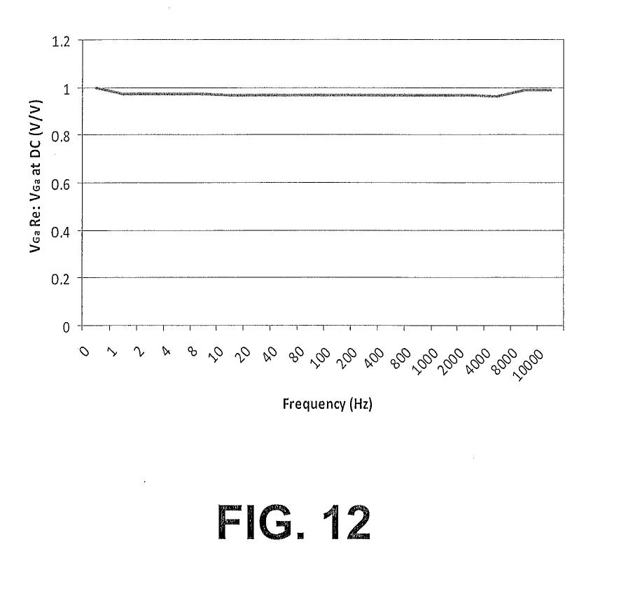

FIG. 12 shows a graph showing the voltage measured across an exemplary Ga-filled liquid metal lead 100 (V_{Ga}) of the present disclosure, referenced to DC (V_{Ga}). The normalized voltage was near unity at all frequencies of interest, indicating that exemplary lead 100 behaved as low impedance resistive element.

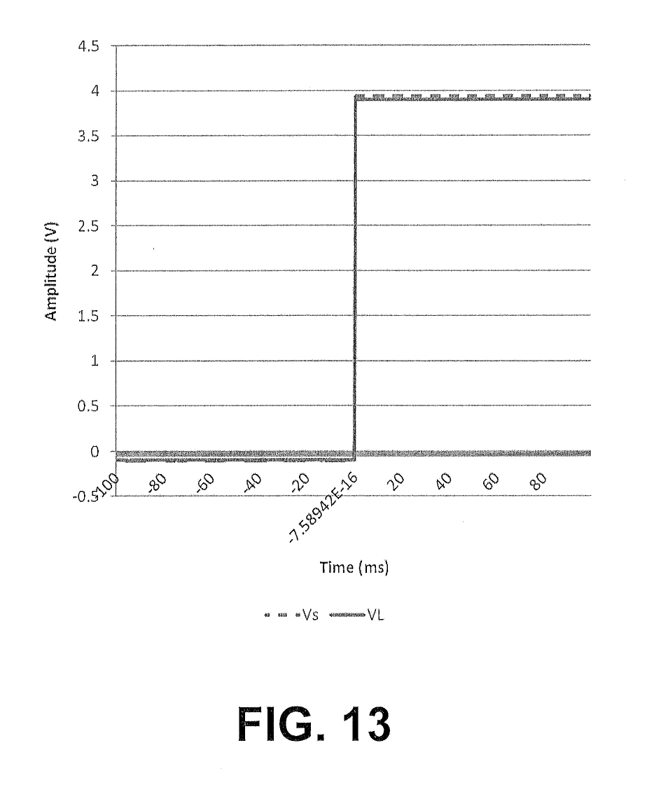

The transient and steady state response of the aforementioned lead 100 was further examined by exciting the liquid lead-R_{L} network with a step voltage input. The circuit was excited with a 4V step input and the output was measured across the load resistor to display any distortive effects from the lead 100. The measured voltage across R_{L} almost exactly mirrored the input voltage, as shown in the graph within FIG. 13, indicating again that the impedance of lead 100 does not introduce any significant phase or amplitude distortion on the signal delivered to a load.

The graph shown in FIG. 13 demonstrates the step response of the exemplary liquid metal lead 100 of the present disclosure in series with load resistor. The output voltage, V_{L}, was measured across the load resistance of 10.5Ω and compared to the input voltage V_{S}.

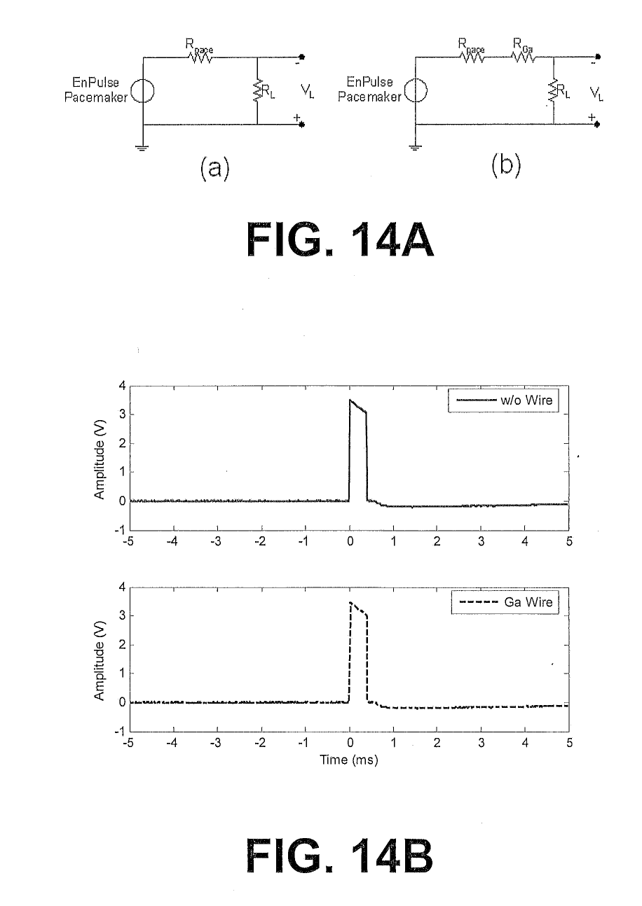

To further test the performance of exemplary lead 100 of the present disclosure with a conventional implantable biomedical device, lead 100 was connected to an implantable cardiac pacemaker (an exemplary biomedical device 300 of the present disclosure). The pacemaker (Medtronic Enpulse Dual Chamber Rate Response Pacemaker) produced a 400 μs, 3.5V quasi-rectangular pulse with a 1 Hz repetition rate. The pacemaker was connected to a conventional bipolar lead (resistance, R_{pace}=23.4Ω) and the device output was measured across a 509Ω load resistance (R_{L}). Then, the liquid wire lead 100 was connected in series with the pacing lead and the load resistance, as shown in the diagrams depicted in FIG. 14A. It was expected that, because the lead 100 was previously demonstrated to be low impedance resistive element, the output from the conventional lead in series with the lead 100 of the present disclosure would be a faithful reproduction of the signal with the conventional lead alone.

FIG. 14A shows an exemplary set of schematics of circuits used to measure the effects of an exemplary lead 100 of the present disclosure on output from (a) a cardiac pacemaker (an exemplary biomedical device 300) with conventional lead, and (b) a cardiac pacemaker with a conventional lead in series with a liquid metal lead 100 of the present disclosure. In such an arrangement, the pacemaker pulse measurement with the liquid wire (lead 100) resulted in an accurate reproduction of an identical signal sent through a conventional lead alone, as shown in the graphs in FIG. 14B. The graph shown in FIG. 14B demonstrates pulse measurements from a pacemaker with a conventional lead (w/o wire) and with a conventional lead in series with a liquid metal lead 100 of the present disclosure (Ga Wire). The pulse of the conventional lead alone was cross-correlated with the pulse measurement with the exemplary lead 100 of the present disclosure.

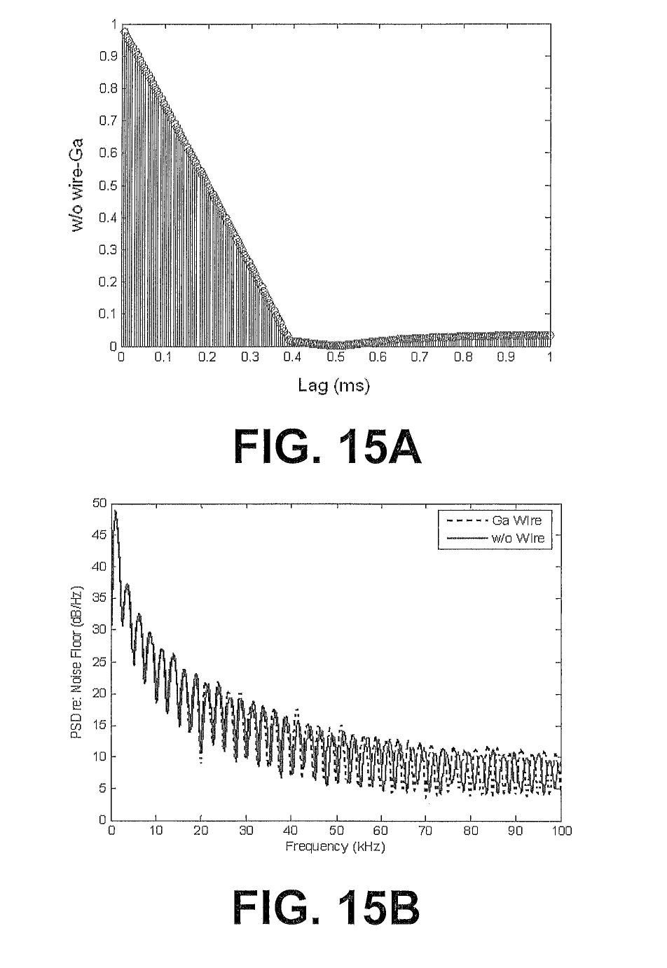

The resultant cross-correlation coefficient had a unity value at a lag value of 0, indicating that there was no delay or waveform distortion due to the presence of the liquid metal conductor, as shown in the graph shown in FIG. 15A. The graph shown in FIG. 15A displays a correlation from pulse measurement with conventional lead alone cross-correlated with pulse measurement from conventional lead in series with liquid metal lead 100 of the present disclosure. Additionally, the power spectra of the pulse measurements with and without the liquid metal lead 100 were highly similar, as shown in the graph in FIG. 15B, which shows a power spectral density estimates from pacemaker with conventional lead (w/o wire) and with conventional lead in series with liquid metal lead 100 of the present disclosure (Ga Wire).

From these measurements, the exemplary unipolar liquid metal lead 100 used in the foregoing tests referenced above shows a very low impedance resistive element, with lead 100 showing the ability to behave as a liquid wire. The exemplary lead 100 embodiment displays operates to any phase or amplitude distortion that occurs with signal transmission. The prototype liquid metal lead 100 was successfully incorporated with a conventional implantable stimulator, namely a cardiac pacemaker, proving that a refined version of a lead 100 could have some biomedical utility without sacrificing the integrity of signal transmission.

### EXAMPLE 2

According to at least one exemplary embodiment of a lead 100 of the present disclosure, the biocompatibility of liquid Ga for use as an electrically conductive composition 200 within a biomedical device lead 100 was examined by exposure of Ga to cells in vitro. Cells, including human umbilical vein endothelial cells (HUVECs), were cultured on tissue culture plastic in fully supplemented growth media. The cells were cultured at two densities, namely 170E3 cell/well and 85E3 cells/well, and Ga droplets were added to each well to determine whether acute Ga exposure would affect cell viability and proliferation (control cells did not receive Ga droplets). While such exposure is not representative of any immunological response to implanted Ga, this result suggests that in vitro exposure will have no effect on cell survival or growth. The cells tested according to at least one exemplary embodiment were in direct contact with sterilized liquid Ga for 24 h and 48 h in a 37° C., 5% CO₂ high humidity incubators.

The cells were then microscopically examined post-Ga exposure. Aside from regions where the liquid metal was initially deposited on the cell wells, there was no evident adverse effect of Ga on the cell monolayers. Cells in direct contact and proximate to the Ga droplets appeared normal and healthy.

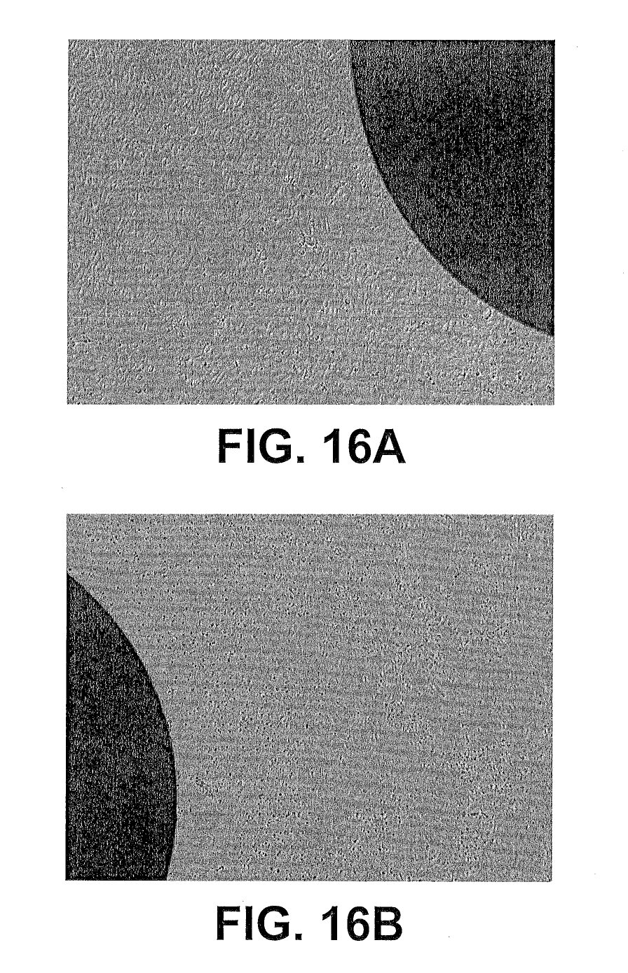

FIGS. 16A and 16B show microscopic images of cells (using a 4× objective) cultured at 170E3 with exposure to Ga (dark regions) at 24 hours (FIG. 16A) and 48 hours (FIG. 16B) post exposure. The cells were detached and counted to quantify the effects of the Ga. These results, in conjunction with the microscopic images shown in FIGS. 16A and 16B, indicate that acute exposure to Ga did not have an adverse effect on human endothelial cells in vitro, which indicates compatibility with a biological system and demonstrating an appropriate conductor material for an implantable lead.

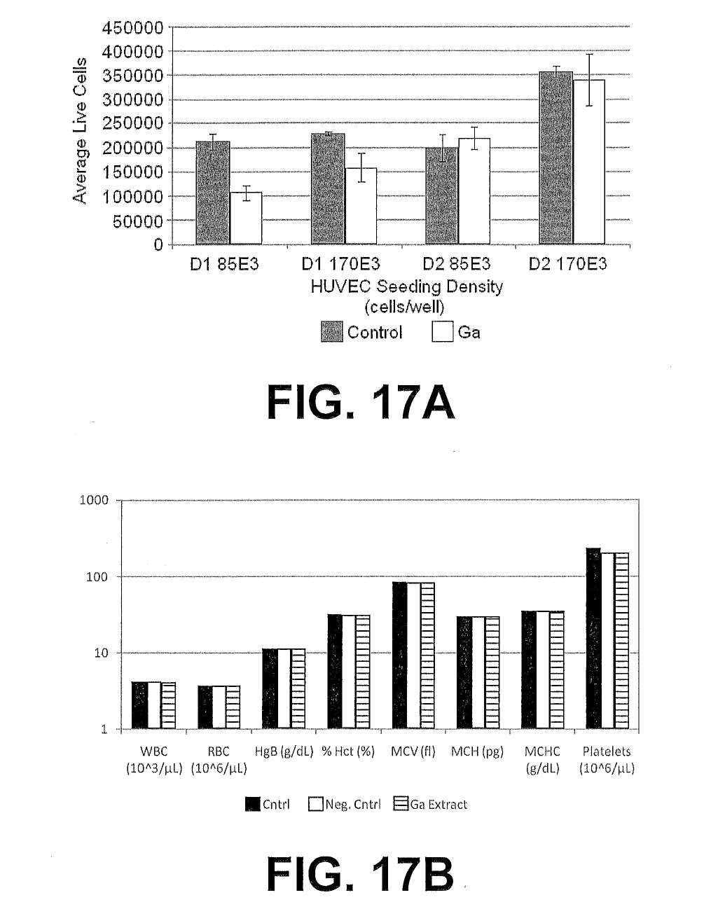

FIG. 17A shows a chart displaying mean cell counts (n=3) from cells exposed to Ga (identified as “Ga”) compared to controls (identified as “Control”). Mean values were compared using Student's T-test with α=0.05. Except for one point at day 1, there were no differences found with Ga, indicating no toxicity.

Further, it has been found that when using a lead 100 according to at least one embodiment of the present disclosure, lead 100 is compatible with magnetic resonance imaging (“MRI”). According to at least one exemplary embodiment of a lead 100 of the present disclosure, lead 100 did not distort or reduce the ability to image surrounding tissues or lead 100 itself, and is thereby compatible with allowing imaging of a patient having a lead 100 of the present disclosure without degrading or otherwise interfering with the imaging process.

Additional biocompatibility testing was conducted in coordination with ToxiKon, Inc., a medical device and biocompatibility testing company. In vitro and in vivo compatibility tests were performed to assess the possible inflammatory or hemolytic effects that could occur in the event of gallium or a gallium alloy, such as Galinstan, leaking from a liquid metal lead in situ.

For such studies, liquid gallium was intracutaneously injected into rabbits to examine the acute inflammatory response to gallium exposure. The study was conducted in accordance with ISO 10993-10, 2002, Biological Evaluation of Medical Devices—Part 10 and ISO 10993-12, 2007, Biological Evaluation of Medical Devices—Part 12. Two rabbits, one male and one female, received intracutaneous injections of 40 μL of liquid gallium (heated to 37° C.) or 0.9% saline solution (control). The gallium was injected on the rabbits' anterior sides, while the control material was injected on the posterior sides. The injection sites were marked with histology ink. The animals were weighed immediately prior to injections and 72 hours post-injection. Response was assessed by total body temperature and examination of the injection sites for erythema and edema. Observations were scored according to the Classification System for Scoring Skin Reactions (Draize Scale). Measurements and photographs of the injection sites were obtained immediately pre-dose, post-dose (0 h) and at 24 hours, 48 hours, and 72 hours post dose.

At the end of the observation period, the rabbits were sacrificed by injection of a barbiturate. The injection sites were excised and fixed in 10% neutral buffered formalin. After macroscopic examination for inflammation, encapsulation, hemorrhage, necrosis, and discoloration, the tissues were sectioned into histological slides. Slides were stained with hematoxylin and eosin (H&E). A ToxiKon pathologist examined the slides and provided a narrative report regarding the injected test article.

The animals' temperature variations throughout the study were minor. The male's pre-dose temperature was 39.0° C., mean post-dose temperature was 39.2° C.±0.320° C. The female's pre-dose temperature was 39.4° C., mean post-dose temperature was 39.5° C. ±0.208° C. The male's weight decreased by 110 mg over the post-injection time, while the female's weight increased by 80 mg. There was no evidence of erythema, edema, or necrosis at any time point or injection site in either animal: The overall mean scores for the gallium injection and control injection sites were both 0.0±0.0. Macroscopic examination of the tissue sections did not exhibit gross evidence of inflammation, encapsulation, hemorrhage, necrosis, or discoloration. However, microscopic examination in gallium tissue samples from both animals displayed punctate black particles near the injection sites.

Whole blood samples were directly exposed to samples of liquid Ga to demonstrate that Ga does not destroy red blood cells. This study was performed in accordance with the following references: ASTM-F756-08, Standard Practice for Assessment of Hemolytic Properties of Materials, 2008, ASTM F619-03, Standard Practice for Extraction of medical Plastics, 2008, ISO 10993-12, 2007, ISO 10993-4, 2002, Biological Evaluation of Medical Devices—Part 4: Selection of Tests for Interactions with Blood. 1.4 g of liquid gallium (37° C.) was added to a vial of 7 mL of PBS. Negative and positive controls contained plastic or Buna-N-Rubber in PBS, respectively. An untreated control consisted of 7 mL of PBS alone. 1 mL of whole rabbit blood was added to each vial and maintained at 37° C. for 3 hours. At the end of the incubation period, the vials were centrifuged, and the supernatant transferred to new vials at 1 mL/vial. The supernatant samples were mixed with 1 mL of Drabkin's reagent. Hemoglobin concentration was spectrophotometrically measured at 540 nm and compared to a hemoglobin standard curve. The release of hemoglobin, i.e., increased hemoglobin in the supernatant, was indicative of red blood cell lysis.

The percent hemolysis of the gallium samples were 0.00% above the level of hemolysis exhibited by the negative control, indicating that direct, acute exposure to gallium is not hemolytic.

An in-vitro hemocompatibility assay was also performed to ensure that gallium does not adversely affect the cellular components of the blood. Extracts of gallium were created by boiling liquid gallium in 0.9% saline solution for 1 hour (200 mg gallium per mL of saline solution). A negative control was created by boiling a plastic sample in saline solution under similar conditions (3 cm³ of sample per mL of saline). Whole human blood was collected and complete blood counts (CBC), platelets, hematocrit, and platelet indices were quantified. In three tubes, gallium extract and blood were mixed (1:9 extract-blood ratio). Negative and untreated control extracts were mixed at the same ratios (three tubes per condition). The tubes were incubated in a warm water bath at 37° C. for 1 hour. Post incubation, CBC platelets, hematocrit, and erythrocyte indices were determined.

The mean and standard deviation (n=3) of the gallium extracts and each of the control extracts were calculated and compared using ANOVA (α=0.05). For each of the hematological parameters, there was not a statistically significant difference between the gallium extracts and the negative or untreated controls as shown in the chart shown in FIG. 17B. As referenced therein, abbreviations/codings refer to mean blood content counts (n=3) for untreated controls (Cntrl), negative controls (Neg. Cntrl), and gallium extract samples (Ga Extract). The blood contents of interest included WBC (white blood cell count), RBC (red blood cell count), HgB (hemoglobin concentration), Hct (hematocrit), MCV (mean corpuscular volume), MCH (mean corpuscular hemoglobin), MCHC (mean corpuscular hemoglobin concentration), and Platelets (platelet count).

The foregoing tests indicate that liquid gallium is well-tolerated both in vitro and in vivo. Intracutaneous infusion of liquid gallium did not elicit severe inflammatory responses after three days of exposure. The macroscopic response of tissues was equivalent to the response of saline injections. Gallium was also demonstrated to be compatible with blood in both direct and indirect exposure conditions.

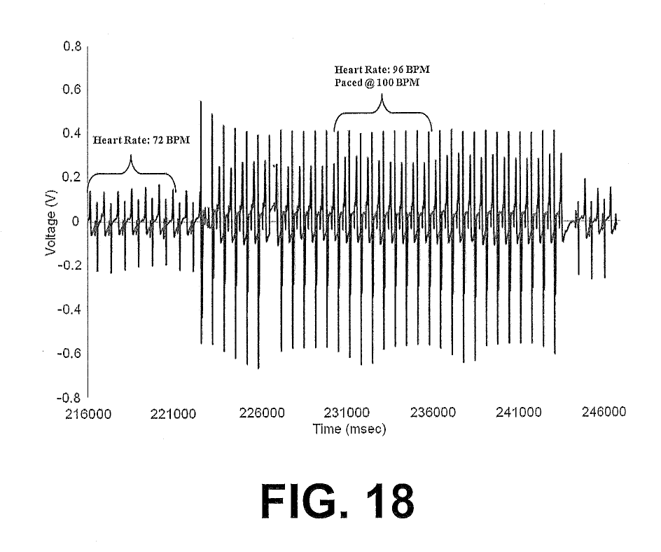

An initial study was also performed to demonstrate that a pacing lead containing liquid gallium as a sole conductor (an exemplary lead 100 of the present disclosure) can successfully stimulate myocardial contractions. The stimulatory capability of a unipolar lead 100 prototype was tested in an open chest swine model, whereby lead 100 was connected to a pacemaker (Medtronic E2DR01 Dual Chamber Rate Responsive Pacemaker) operating in VOO mode. The terminal electrode of lead 100 was sutured to the epicardium of the right and the left ventricles of the test subject, and the subject's cardiac electrogram was monitored via a sensing electrode placed in the epicardium. The swine's intrinsic heart was measured at 72 bpm. When the pacemaker was activated at a rate of 100 bpm, capture was achieved as shown in the cardiac electrogram shown in FIG. 18. Capture beats were elicited in both right and ventricular stimulation, demonstrating that various leads 100 of the present disclosure can be used to transmit electrical signals from a pacemaker to stimulate the myocardium.

While various embodiments of implantable biomedical device leads comprising liquid conductors and methods for using the same have been described in considerable detail herein, the embodiments are merely offered by way of non-limiting examples of the disclosure described herein. It will therefore be understood that various changes and modifications may be made, and equivalents may be substituted for elements thereof, without departing from the scope of the disclosure. Indeed, this disclosure is not intended to be exhaustive or to limit the scope of the disclosure.

Further, in describing representative embodiments, the disclosure may have presented a method and/or process as a particular sequence of steps. However, to the extent that the method or process does not rely on the particular order of steps set forth herein, the method or process should not be limited to the particular sequence of steps described. Other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A lead for use in a biomedical application, the lead comprising:
a lead body having a distal end and a proximal end, the lead body defining a first interior lumen therethrough;
a solid conductor positioned within the first interior lumen of the lead body; and
an electrically conductive composition positioned within the first interior lumen, the electrically conductive composition comprising a metal in a liquid state at or below about 98° F.;
wherein the solid conductor and the electrically conductive composition are each configured for conductive communication with a distal component of the lead body.

2. The lead of claim 1, wherein the electrically conductive composition is selected from the group consisting of gallium, a gallium-indium alloy, Galinstan, its/their alloys, and combinations thereof.

3. The lead of claim 1, wherein the lead body comprises a non-conductive material selected from the group consisting of silicone, polyurethane, polytetrafluoroethylene (PTFE), ethylene tetraflurooethylene (ETFE), polyether ether ketone (PEEK), perfluroralkoxyethylene-tetrafluoroethylene (PFA), fluorinated ethylene propylene (FEP), polyvinylidene fluoride (PVDF), polyamide, parylene, nylon, co-extrusions thereof, and combinations thereof.

4. The lead of claim 1, wherein the first interior lumen is filled with the electrically conductive composition under vacuum or under atmospheric pressure and maintained within the first interior lumen under vacuum or under atmospheric pressure.

5. (canceled)

6. (canceled)

7. The lead of claim 1, further comprising;
a distal component coupled to the lead body at or near the distal end of the lead body, the distal component in conductive communication with the electrically conductive composition and the solid conductor, wherein the distal component is selected from the group consisting of an electrode, a connector, an adapter, a coil, and a closure device.

8. (canceled)

9. The lead of claim 7, wherein the distal component is capable of sealing the distal end of the lead body to prohibit loss of the electrically conductive composition from the first interior lumen and to mitigate entry of bodily fluids into the first interior lumen.

10.-12. (canceled)

13. The lead of claim 1, wherein the solid conductor comprises a conductive material selected from the group consisting of stainless steel, platinum, titanium, silver, tantalum, a nickel-cobalt base alloy, its/their alloys, and combinations thereof.

14. The lead of claim 1, wherein the electrically conductive composition and the solid conductor are capable of transmitting a first electrical signal therethrough.

15. The lead of claim 14, wherein the electrically conductive composition and the solid conductor are capable of transmitting the first electrical signal in a first direction.

16. The lead of claim 1, wherein the electrically conductive composition is capable of transmitting a first electrical signal therethrough, and wherein the solid conductor is capable of transmitting a second electrical signal therethrough; and wherein the lead further comprises:
an insulator positioned between the electrically conductive composition and the solid conductor.

17. (canceled)

18. The lead of claim 1, further comprising:
a second interior lumen defined within the lead body; and
a second solid conductor positioned within the second interior lumen of the lead body, wherein the electrically conductive composition is capable of transmitting a first electrical final therethrough, and wherein the second solid conductor is capable of transmitting either the first electrical signal or a second electrical signal therethrough.

19.-21. (canceled)

22. The lead of claim 1, further comprising:
a proximal component coupled to the lead body at or near the proximal end of the lead body, the proximal component in conductive communication with the electrically conductive composition and the solid conductor; and
a biomedical device coupled to the proximal component, the biomedical device capable of transmitting and or receiving an electrical signal from the electrically conductive composition;
wherein the biomedical device is selected from the group consisting of a cardiac pacemaker, an implantable cardioverter defibrillator, an electrocardiogram (EKG or ECG) device, and electroencephalogram (EEG) device, a neurological stimulator, and a non-neurological stimulator.

23.-53. (canceled)

54. The lead of claim 1, further comprising:
a doped portion at or near the distal end of the lead body, the doped portion comprising a non-conductive material and a conductive material, wherein the conductive material is selected from the group consisting of stainless steel, platinum, titanium, a nickel-cobalt base alloy, carbon black, carbon fiber, nanofiber tubes, polyaniline, polypyrrole, polythiophene, polyethylenedioxythiophene, and poly(p-phenylene vinylene).

55.-60. (canceled)

61. The lead of claim 1, wherein the lead body has a cross-sectional area configured so that one or more conductance pockets are defined within the first interior lumen upon compression of the lead body.

62. The lead of claim 1, wherein the lead body comprises one or more relatively thinner portions and one or more relatively thicker portions, wherein said one or more relatively thinner portions and the one or more relatively thicker portions define an irregularly-shaped first interior lumen, and wherein when the lead body is compressed, the one or more relatively thicker portions define one or more conductance pockets therein.

63.-72. (canceled)

73. The lead of claim 1, wherein the first interior lumen comprises a configuration selected from the group consisting of a straight configuration, a twisted configuration, and a coiled configuration.

74.-83. (canceled)

84. The lead of claim 1, wherein the distal component is at least partially coated with a fibrosis-enhancing substance, the fibrosis-enhancing substance capable of facilitating localized fibrosis at or near the distal component upon connection of the lead to a tissue within a patient's body to reduce the likelihood of unintended disconnection of the lead from the tissue.

85.-89. (canceled)

90. The lead of claim 1, wherein the lead body is sufficiently flexible and the electrically conductive composition is sufficiently pliable so to reduce a likelihood of tissue perforation within a patient's body when the lead is positioned within the patient's body and coupled to the tissue as compared to a traditional lead having a solid conductor.

91.-98. (canceled)

99. A system, comprising:
a lead for use in a biomedical application, the lead comprising:
a lead body having a distal end and a proximal end, the lead body defining a first interior lumen therethrough;
a solid conductor positioned within the first interior lumen of the lead body; and
an electrically conductive composition positioned within the first interior lumen, the electrically conductive composition in a liquid or gel state at or below about 98° F.; and
a biomedical device coupled to the lead, the biomedical device capable of transmitting and/or receiving an electrical signal from the electrically conductive composition and the solid conductor within the lead;
wherein the solid conductor and the electrically conductive composition are each configured for conductive communication with the biomedical device

100. A lead for use in a biomedical application, the lead comprising:
a lead body having a distal end and a proximal end, the lead body defining a first interior lumen therethrough;
an electrically conductive composition positioned within the first interior lumen, the electrically conductive composition comprising a metal in a liquid state at or below about 98° F., the electrically conductive composition selected, from the group consisting of gallium, a gallium-indium alloy, Galinstan, its/their alloys, and combinations thereof;
a solid conductor positioned within the first interior lumen of the lead body; a distal component coupled to the lead body at or near the distal end of the lead body, the distal component in conductive communication with the electrically conductive composition and the solid conductor, the distal component capable of sealing the distal end of the lead body; and
a proximal component coupled to the lead body at or near the proximal end of the lead body, the proximal component in conductive communication with the electrically conductive composition and the solid conductor, the proximal component capable of sealing the proximal end of the lead body;
the lead capable of transmitting a first electrical signal from the proximal component, through the electrically conductive composition and the solid conductor, and to the distal component.

101.-104. (canceled)
